(19) United States
(12) Patent Application Publication
(10) Pub. No.: US 2010/0173313 A1
(43) Pub. Date: Jul. 8, 2010

(54) BIOMARKERS OF INFLAMMATION

(75) Inventors: Gretchen BAIN, San Diego, CA (US); John Howard HUTCHINSON, La Jolla, CA (US); Brian Andrew STEARNS, San Diego, CA (US); Kevin Murray SCHAAB, Spring Valley, CA (US); Jillian F. EVANS, San Diego, CA (US)

Correspondence Address: WILSON, SONSINI, GOODRICH & ROSATI, 650 PAGE MILL ROAD, PALO ALTO, CA, 94304-1050, US

(73) Assignee: AMIRA PHARMACEUTICALS, INC., San Diego, CA (US)

(21) Appl. No.: 12/684,450

(22) Filed: Jan. 8, 2010

Related U.S. Application Data
(60) Provisional application No. 61/143,349, filed on Jan. 8, 2009.

(51) Int. Cl. C12Q 1/06; C12Q 1/02; C12Q 1/68

(52) U.S. Cl. 435/6; 435/29; 435/39

(57) ABSTRACT

Methods for the detection of DP₂ receptors in biological samples is disclosed. The induction and detection of DP₂ receptors expressed on activated human neutrophils is presented. Also presented are diagnostic kits for the detection of DP₂ receptors in biological samples.

### RELATED APPLICATIONS

This application claims the benefit of U.S. provisional patent application No. 61/143,349 entitled “BIOMARKERS OF INFLAMMATION” filed on Jan. 8, 2009, which is incorporated by reference in its entirety.

### FIELD OF THE INVENTION

Biomarkers of inflammation include inflammatory cells types, protein receptors, inflammatory molecules, and other indicators involved in inflammatory pathways. Neutrophils are an example of an inflammatory cell. The chemoattractant receptor-homologous molecule expressed on T_{H}2 cells (CRTH2; also known as DP2 or DP₂; all of these terms are used interchangeably herein) mediates some of the biological effects of prostaglandin D₂ (PGD₂). Methods for detecting DP₂ receptors expressed on neutrophils are described.

### BACKGROUND OF THE INVENTION

Prostaglandins are derived from the metabolism of arachidonic acid by the action of cyclooxygenase enzymes and downstream synthases. Prostaglandins have a diverse range of physiological and pathological activities and have a well recognized role in pain and inflammation. PGD₂ is an acidic lipid mediator derived from the metabolism of arachidonic acid by cyclooxygenases and PGD₂ synthases. PGD₂ binds to a number of receptors, which include the thromboxane-type prostanoid (TP) receptor, PGD₂ receptor (DP, also known as DP₁) and chemoattractant receptor-homologous molecule expressed on T_{H}2 cells (CRTH2; also known as DP₂). The activation of DP₂ on inflammatory cells exacerbates inflammatory diseases in animal models. Inflammatory diseases or conditions include respiratory diseases or conditions and allergic diseases or conditions. Neutrophilic inflammation plays a role in many diseases or conditions.

### SUMMARY OF THE INVENTION

In one aspect, provided is a diagnostic test. In one aspect, the diagnostic test assesses whether a mammal is experiencing a disease or condition wherein neutrophilic inflammation is a component of the pathophysiology of the disease or condition. In one aspect, the detection of DP₂ receptors on neutrophils provides a diagnostic test for neutrophilic inflammation. In one aspect, the detection of DP₂ receptors on neutrophils is used to: (a) monitor disease activity and/or provide mechanism of action of drug candidate(s); (b) predict rapid disease progression and/or indicate the onset and resolution of an acute inflammatory exacerbation; (c) prescribe targeted and individualized therapies and/or evaluate the response to treatment with a DP₂ antagonist; (d) means of patient stratification for clinical trials involving DP₂ antagonists; and/or (e) serve as candidate surrogate endpoints in early-phase clinical trials.

In one aspect, the diagnostic test presented herein provide methods for: (a) diagnosing humans with inflammatory diseases or condition; (b) preventing further exacerbations of symptoms associated with inflammatory diseases or conditions; and/or (c) treating inflammatory diseases or condition in humans with at least one DP₂ antagonist. In one aspect, the diagnostic test presented herein provide a method for determining a therapeutically effective amount of a DP₂ antagonist to be adminstered to a human.

In one aspect, described herein is a method of identifying a human for treatment with a DP₂ antagonist comprising: detecting DP₂ receptors in a transformed biological sample from a human using at least one analytical instrument.

In one aspect, the method comprises detecting DP₂ receptors expressed on polymorphonuclear leukocytes in the transformed biological sample from the human using at least one analytical instrument.

In one aspect, the method comprises detecting DP₂ receptors expressed on neutrophils in the transformed biological sample from the human using at least one analytical instrument.

In some embodiments, the human has at least one symptom of an inflammatory disease or condition. In one aspect, the inflammatory disease or condition is a respiratory disease or condition, or an allergic disease or condition. In some embodiments, the inflammatory disease or condition is selected from asthma, rhinitis, allergic conjunctivitis, chronic obstructive pulmonary disease (COPD), pulmonary hypertension, interstitial lung fibrosis, adult respiratory distress syndrome, airway constriction, mucus secretion, nasal congestion, sinusitis, and chronic cough. In other embodiments, the inflammatory disease or condition is selected from allergic asthma, non-allergic asthma, acute severe asthma, chronic asthma, clinical asthma, nocturnal asthma, allergen-induced asthma, aspirin-sensitive asthma, exercise-induced asthma, child-onset asthma, adult-onset asthma, cough-variant asthma, occupational asthma, steroid-resistant asthma, seasonal asthma, chronic obstructive pulmonary disease, pulmonary hypertension, and interstitial lung fibrosis.

In some embodiments, the inflammatory disease or condition is insensitive to treatment with corticosteroids.

In one aspect, the inflammatory disease or condition is an inflammatory disease or condition wherein neutrophils contribute to the symptomology of the inflammatory disease or condition.

In one aspect, the transformed biological sample from the human is a transformed biological fluid or transformed biological tissue sample. In some embodiments, the transformed biological sample from the human comprises polymorphonuclear leukocytes. In some embodiments, the transformed biological sample from the human comprises neutrophils.

In one aspect, the transformed biological sample from the human is from an area of disease activity.

In some embodiments, the transformed biological sample from the human is a transformed sputum sample, a transformed bronchoalveolar lavage sample, a transformed nasal lavage sample, a transformed pleural effusion sample, a transformed fine needle aspirate sample from the lung, a transformed lymph node sample from the lung, or a transformed lung wash sample.

In some embodiments, the method of detecting further comprises classifying the inflammatory disease or condition as a PGD₂-dependent or a PGD₂-mediated disease or condition based on the detection of DP₂ receptors in the transformed biological sample from the human.

In some embodiments, the method of detecting further comprises classifying the inflammatory disease or condition as a PGD₂-dependent or a PGD₂-mediated disease or condition based on the detection of DP₂ receptors expressed on neutrophils in the transformed biological sample from the human.

In some embodiments, the method of detecting further comprises classifying the human as eligible to receive therapy for the inflammatory disease or condition based on the detection of DP₂ receptors in the transformed biological sample from the human.

In some embodiments, the method of detecting further comprises classifying the human as eligible to receive therapy for the inflammatory disease or condition based on the detection of DP₂ receptors expressed on neutrophils in the transformed biological sample from the human. In some embodiments, the therapy for the inflammatory disease or condition comprises administration of a DP₂ antagonist to the human.

In one aspect, the DP₂ antagonist is a small molecule DP₂ antagonist.

In one aspect, the DP₂ antagonist is selected from compounds disclosed in U.S. provisional application No. 61/031,310; International patent application no. PCT/US09/35174; U.S. provisional application No. 60/985,919; International patent application no. PCT/US08/82056; U.S. provisional application No. 60/985,913; International patent application no. PCT/US08/82082; U.S. provisional application No. 61/025,597; International patent application no. PCT/US09/32495; International patent application no. PCT/US09/32499; U.S. application Ser. No. 12/362,439; U.S. provisional application No. 61/110,496; U.S. provisional application No. 61/028,804; International patent application no. PCT/US09/33961; U.S. provisional application No. 61/041,869; International patent application no. PCT/US09/38291; U.S. provisional application No. 61/078,311; International patent application no. PCT/US09/49621; International patent application no. PCT/US09/49631; U.S. application Ser. No. 12/497,343; U.S. provisional application No. 61/101,074; International patent application no. PCT/US09/58655; International patent application no. PCT/US09/58663; Ser. No. 12/568,571; U.S. provisional application No. 61/054,093; U.S. provisional application No. 61/107,638; International patent application no. PCT/US09/44219; U.S. provisional application No. 61/075,242; International patent application no. PCT/US09/48327; U.S. provisional application No. 61/101,964; International patent application no. PCT/US09/59256; U.S. provisional application No. 61/103,872; International patent application no. PCT/US09/59891; U.S. provisional application No. 61/115,259; International patent application no. PCT/US09/64630; U.S. provisional application No. 61/112,044; International patent application no. PCT/US09/63439; International patent application no. PCT/US09/63438; U.S. application Ser. No. 12/613,424.

In one aspect, the DP₂ antagonist is selected from: AZD1981, ODC9101 (OC459), OC499, OC1768, OC2125, OC2184, QAV680, MLN6095, AP768, [2′-(3-Benzyl-1-ethyl-ureidomethyl)-6-methoxy-4′-trifluoromethyl-biphenyl-3-yl]-acetic acid, {3-[2-tert-Butylsulfanylmethyl-4-(2,2-dimethyl-propionylamino)-phenoxy]-4-methoxy-phenyl}-acetic acid, TM30642, TM30643, TM30089, TM27632, TM3170.

In one aspect, the DP₂ antagonist is selected from [2′-(3-Benzyl-1-ethyl-ureidomethyl)-6-methoxy-4′-trifluoromethyl-biphenyl-3-yl]-acetic acid, and {3-[2-tert-Butylsulfanylmethyl-4-(2,2-dimethyl-propionylamino)-phenoxy]-4-methoxy-phenyl}-acetic acid.

In one aspect, the transformed biological sample is produced by transforming the DP₂ receptors in a biological sample into DP₂ receptors that are detectable using at least one analytical instrument. In some embodiments, transforming the DP₂ receptors in the biological sample into DP₂ receptors that are detectable using at least one analytical instrument comprises contacting the biological sample from the human with a DP₂ ligand under conditions suitable for ligand-receptor interactions.

In one aspect, the DP₂ ligand is a peptide, a peptidomimetic, an antibody, an aptamer, an oligonucleotide or a small molecule DP₂ antagonist.

In some embodiments, the DP₂ ligand comprises a detectable label.

In some embodiments, the DP₂ ligand comprises a detectable label selected from a fluorescent label, an enzyme label, a radioactive label, a nuclear magnetic resonance active label, a luminescent label, a paramagnetic label, a chromophore label, a biotin tag, and an avidin tag.

In one aspect, the DP₂ ligand comprises a fluorescent label.

In one aspect, the DP₂ ligand is an antibody comprising a fluorescent label.

In some embodiments, the DP₂ ligand is a small molecule DP₂ antagonist comprising a fluorescent label. In some embodiments, the DP₂ ligand is a small molecule DP₂ antagonist comprising a fluorescent label having the following structure:

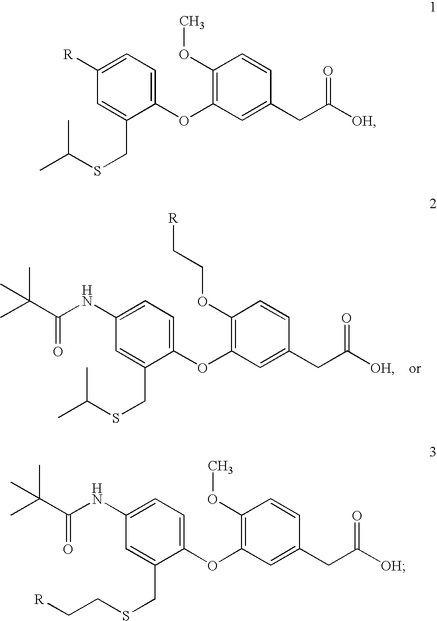

where R is Dansyl-NH—, Texas red-NH—, Alexa Fluor 350-NH—, Alexa Fluor 488-NH—, Alexa Fluor 546-NH—, Alexa Fluor 555-NH—, Alexa Fluor 647-NH—, Cy3-NH—, Cy5-NH—, or Cy7-NH.

In some embodiments, detecting DP₂ receptors in the transformed biological sample from the human further comprises measuring fluorescence from the transformed biological sample after the transformed biological sample is contacted with the DP₂ ligand under conditions suitable for ligand-receptor interactions.

In some embodiments, detecting DP₂ receptors in the transformed biological sample from the human using at least one analytical instrument employs a technique selected from the group consisting of: Fluorescence-activated Cell Sorting (FACS), confocal, western blot, flow cytometry, fluorescence microscopy, scintillation counting, quantititative polymerase chain reaction, Magnetic-activated Cell Sorting (MACS). Other techniques are contemplated.

In some embodiments, the classifying further comprises determining the number of neutrophils that express DP₂ receptors in the biological sample from the human from an area of disease activity.

In some embodiments, the classifying further comprises comparing the number of neutrophils that express DP₂ receptors in the biological sample from the human from the area of disease activity to the number of neutrophils that express DP₂ receptors in a whole blood sample from the human.

In some embodiments, a portion of the transformed biological sample is exposed to an endotoxin prior to detecting DP₂ receptors in the transformed biological sample.

In another aspect, described is a method of identifying a human with an respiratory disease or condition as eligible for treatment with a DP₂ antagonist comprising detecting DP₂ receptors expressed on neutrophils in a transformed biological sample from a human with a respiratory disease or condition using at least one analytical instrument.

In some embodiments, the respiratory disease or condition is selected from asthma, chronic obstructive pulmonary disease (COPD), pulmonary hypertension, interstitial lung fibrosis, adult respiratory distress syndrome, and airway constriction.

In one aspect, the respiratory disease or condition is selected from allergic asthma, non-allergic asthma, acute severe asthma, chronic asthma, clinical asthma, nocturnal asthma, allergen-induced asthma, aspirin-sensitive asthma, exercise-induced asthma, child-onset asthma, adult-onset asthma, cough-variant asthma, occupational asthma, steroid-resistant asthma, seasonal asthma, chronic obstructive pulmonary disease, pulmonary hypertension, and interstitial lung fibrosis.

In certain cases, neutrophils contribute to the symptomology of the respiratory disease or condition.

In certain embodiments, the respiratory disease or condition is insensitive to treatment with corticosteroids.

In one aspect, the biological sample from the human is a biological fluid or biological tissue sample.

In one aspect, the biological sample from the human is from an area of disease activity.

In some embodiments, the biological sample from the human is a biological fluid or biological tissue sample from or around the lungs.

In some embodiments, the biological sample from the human is a sputum sample, a saliva sample, a bronchoalveolar lavage sample, a nasal lavage sample, a pleural effusion sample, a fine needle aspirate sample from the lung, a lymph node sample from the lung, or a lung wash sample.

In certain embodiments, the method further comprises classifying the human as eligible to receive therapy for the respiratory disease or condition based on the detection of DP₂ receptors expressed on neutrophils in the biological sample from the human. In one aspect, the therapy for the respiratory disease or condition comprises administration of a DP₂ antagonist to the human.

In one aspect, the classifying further comprises comparing the number of neutrophils that express DP₂ receptors in the biological sample from the human from the area of disease activity to the number of neutrophils that express DP₂ receptors in a whole blood sample from the human.

In some embodiments, the DP₂ antagonist is a small molecule DP₂ antagonist.

In another aspect, the DP₂ antagonist is selected from AZD1981, ODC9101 (OC459), OC499, OC1768, OC2125, OC2184, QAV680, MLN6095, AP768, [2′-(3-Benzyl-1-ethyl-ureidomethyl)-6-methoxy-4′-trifluoromethyl-biphenyl-3-yl]-acetic acid, {3-[2-tert-Butylsulfanylmethyl-4-(2,2-dimethyl-propionylamino)-phenoxy]-4-methoxy-phenyl}-acetic acid, TM30642, TM30643, TM30089, TM27632, TM3170.

In yet another aspect, the DP₂ antagonist is selected from [2′-(3-Benzyl-1-ethyl-ureidomethyl)-6-methoxy-4′-trifluoromethyl-biphenyl-3-yl]-acetic acid, and {3-[2-tert-Butylsulfanylmethyl-4-(2,2-dimethyl-propionylamino)-phenoxy]-4-methoxy-phenyl}-acetic acid.

In one embodiment, detecting DP₂ receptors in the biological sample from the human using at least one analytical instrument comprises transforming the DP₂ receptors in the biological sample into DP₂ receptors that are detectable using the least one analytical instrument.

In certain embodiments, transforming the DP₂ receptors in the biological sample into DP₂ receptors that are detectable using at least one analytical instrument comprises contacting the biological sample from the human with a DP₂ ligand under conditions suitable for ligand-receptor interactions.

In some embodiments, the DP₂ ligand is a peptide, a peptidomimetic, an antibody, an aptamer, an oligonucleotide or a small molecule DP₂ antagonist.

In one aspect, the DP₂ ligand comprises a detectable label.

In some embodiments, the DP₂ ligand comprises a detectable label selected from a fluorescent label, an enzyme label, a radioactive label, a nuclear magnetic resonance active label, a luminescent label, a paramagnetic label, a chromophore label, a biotin tag, and an avidin tag.

In certain embodiments, the DP₂ ligand comprises a fluorescent label.

In one aspect, the DP₂ ligand is an antibody comprising a fluorescent label. In a particular embodiment, the antibody is a DP2 antibody.

In one aspect, the DP₂ ligand is a small molecule DP₂ antagonist comprising a fluorescent label.

In some embodiments, detecting DP₂ receptors in the biological sample from the human further comprises measuring fluorescence from the biological sample after the biological sample is contacted with the DP₂ ligand under conditions suitable for ligand-receptor interactions.

In certain embodiments, detecting DP₂ receptors in the biological sample from the human using at least one analytical instrument employs a technique selected from the group consisting of: Fluorescence-activated Cell Sorting (FACS), confocal, western blot, flow cytometry, fluorescence microscopy, scintillation counting, quantititative polymerase chain reaction, magnetic-activated Cell Sorting (MACS). Other techniques are contemplated.

In some embodiments, a portion of the transformed biological sample is exposed to an endotoxin prior to detecting DP₂ receptors in the transformed biological sample. In some embodiments, a portion of the transformed biological sample is exposed to an endotoxin and/or neutrophil chemoattractants/activating ligands prior to detecting DP₂ receptors in the transformed biological sample.

In another aspect, described is a method of identifying a human for treatment with a DP₂ antagonist comprising: transforming DP₂ receptors in a biological sample from a human into DP₂ receptors that are detectable using at least one analytical instrument; and a) identifying cells in the biological sample from a human that express the DP₂ receptor using at least one analytical instrument; b) quantifying the number of cells in the biological sample from the human that express the DP₂ receptor using at least one analytical instrument; or c) identifying and quantifying the number of cells in the biological sample from the human that express the DP₂ receptor using at least one analytical instrument.

In some embodiments, transforming DP₂ receptors in a biological sample from a human into DP₂ receptors that are detectable using at least one analytical instrument comprises contacting the biological sample from the human with a DP₂ ligand under conditions suitable for ligand-receptor interactions.

In one aspect, the cells that are identified, quantified, or identified and quantified that express the DP₂ receptor using at least one analytical instrument are neutrophils.

In some embodiments, the human has at least one symptom of an inflammatory disease or condition.

In some embodiments, the inflammatory disease or condition is a respiratory disease or condition, or an allergic disease or condition.

In some embodiments, the inflammatory disease or condition is selected from asthma, chronic obstructive pulmonary disease (COPD), pulmonary hypertension, interstitial lung fibrosis, adult respiratory distress syndrome, and airway constriction.

In one aspect, the inflammatory disease or condition is selected from allergic asthma, non-allergic asthma, acute severe asthma, chronic asthma, clinical asthma, nocturnal asthma, allergen-induced asthma, aspirin-sensitive asthma, exercise-induced asthma, child-onset asthma, adult-onset asthma, cough-variant asthma, occupational asthma, steroid-resistant asthma, seasonal asthma, chronic obstructive pulmonary disease, pulmonary hypertension, and interstitial lung fibrosis.

In one aspect, neutrophilic inflammation contributes to the pathophysiology of the inflammatory disease or condition, respiratory disease or condition, or allergic disease or condition.

In one aspect, the biological sample from the human is a biological fluid or biological tissue sample. In one aspect, the biological sample from the human comprises neutrophils. In one aspect, the biological sample from the human is from an area of disease activity.

In some embodiments, the biological sample from the human is a sputum sample, a bronchoalveolar lavage sample, a nasal lavage sample, a pleural effusion sample, a fine needle aspirate sample from the lung, a lymph node sample from the lung, or a lung wash sample.

In certain embodiments, the method further comprises classifying the human as eligible to receive therapy for the inflammatory disease or condition based on the detection of DP₂ receptors expressed on neutrophils in the biological sample from the human. In one aspect, the therapy for the inflammatory disease or condition comprises administration of a DP₂ antagonist to the human. In one aspect, the DP₂ antagonist is a small molecule DP₂ antagonist.

In one aspect, the DP₂ antagonist is selected from AZD1981, ODC9101 (OC459), OC499, OC1768, OC2125, OC2184, QAV680, MLN6095, AP768, [2′-(3-Benzyl-1-ethyl-ureidomethyl)-6-methoxy-4′-trifluoromethyl-biphenyl-3-yl]-acetic acid, {3-[2-tert-Butylsulfanylmethyl-4-(2,2-dimethyl-propionylamino)-phenoxy]-4-methoxy-phenyl}-acetic acid, TM30642, TM30643, TM30089, TM27632, TM3170.

In some embodiments, the DP₂ ligand is a peptide, a peptidomimetic, an antibody, an aptamer, an oligonucleotide or a small molecule DP₂ antagonist.

In one aspect, the DP₂ ligand comprises a detectable label.

In certain embodiments, the DP₂ ligand comprises a detectable label selected from a fluorescent label, an enzyme label, a radioactive label, a nuclear magnetic resonance active label, a luminescent label, a paramagnetic label, a chromophore label, a biotin tag, and an avidin tag.

In one aspect, the DP₂ ligand comprises a fluorescent label.

In one aspect, the DP₂ ligand is an antibody comprising a fluorescent label.

In another aspect, the DP₂ ligand is a small molecule DP₂ antagonist comprising a fluorescent label.

In some embodiments, the method employs a protein detection technique selected from the group consisting of: Fluorescence-activated Cell Sorting (FACS), confocal, western blot, flow cytometry, fluorescence microscopy, or a DP2 messenger RNA detection by for example quantitative polymerase chain reaction.

In one aspect, the classifying further comprises comparing the number of neutrophils that express DP₂ receptors in the biological sample from the human from the area of disease activity to the number of neutrophils that express DP₂ receptors in a whole blood sample from the human.

In some embodiments, a portion of the biological sample is exposed to an endotoxin prior to detecting DP₂ receptors in the biological sample.

Also provided is a method of monitoring the clinical efficacy of a DP₂ antagonist in a human comprising comparing: (1) the detection of DP₂ receptors in a first biological sample from a human using at least one analytical instrument prior to the administration of a DP₂ antagonist to the human, with (2) the detection of DP₂ receptors in a second biological sample from the human using at least one analytical instrument after the administration of the DP₂ antagonist to the human; wherein the first biological sample and the second biological sample are the same, and the first biological sample and the second biological sample comprise polymorphonuclear leukocytes.

In some embodiments, the method of monitoring the clinical efficacy of a DP₂ antagonist in a human comprises comparing: (1) the detection of DP₂ receptors expressed on polymorphonuclear leukocytes in a first biological sample from a human using at least one analytical instrument prior to the administration of a DP₂ antagonist to the human with (2) the detection of DP₂ receptors expressed on polymorphonuclear leukocytes in a second biological sample from the human using at least one analytical instrument after the administration of the DP₂ antagonist to the human.

In some embodiments, the method of monitoring the clinical efficacy of a DP₂ antagonist in a human comprises comparing: (1) the detection of DP₂ receptors expressed on neutrophils in a first biological sample from a human using at least one analytical instrument prior to the administration of a DP₂ antagonist to the human with (2) the detection of DP₂ receptors expressed on neutrophils in a second biological sample from the human using at least one analytical instrument after the administration of the DP₂ antagonist to the human.

In one aspect, the first biological sample and the second biological sample comprise neutrophils.

In one aspect, the human has at least one symptom of an inflammatory disease or condition.

In some embodiments, the inflammatory disease or condition is selected from asthma, rhinitis, allergic conjunctivitis, atopic dermatitis, chronic obstructive pulmonary disease (COPD), pulmonary hypertension, interstitial lung fibrosis, arthritis, allergy, psoriasis, inflammatory bowel disease, adult respiratory distress syndrome, myocardial infarction, aneurysm, stroke, cancer, wound healing, endotoxic shock, pain, inflammatory conditions, eosinophilic esophagitis, eosinophil-associated gastrointestinal disorders (EGID), idiopathic hypereosinophilic syndrome, otitis, airway constriction, mucus secretion, nasal congestion, urticaria, sinusitis, uveitis, angioedema, anaphylaxia, chronic cough and Churg Strauss syndrome.

In some aspects, the inflammatory disease or condition is a respiratory disease or condition.

In some other aspects, the inflammatory disease or condition is an allergic disease or condition.

In certain embodiments, the inflammatory disease or condition is an inflammatory disease or condition wherein neutrophils contribute to the symptomology of the inflammatory disease or condition.

In some embodiments, the inflammatory disease or condition is insensitive to treatment with corticosteroids.

In one aspect, the first biological sample and the second biological sample are a biological fluid or a biological tissue sample.

In certain embodiments, the first biological sample and the second biological sample are from an area of disease activity.

In some embodiments, the first biological sample and the second biological sample are selected from a sputum sample, a bronchoalveolar lavage sample, a nasal lavage sample, a pleural effusion sample, a fine needle aspirate sample from the lung, a lymph node sample from the lung, and a lung wash sample.

In one aspect, a reduction of the number of polymorphonuclear leukocytes expressing the DP₂ receptor that are detected in the second biological sample relative to the number of polymorphonuclear leukocytes expressing the DP₂ receptor that are detected in the first biological samples indicates a positive response to the DP₂ antagonist.

In certain embodiments, a reduction of the number of neutrophils expressing the DP₂ receptor that are detected in the second biological sample relative to the number of neutrophils expressing the DP₂ receptor that are detected in the first biological samples indicates a positive response to the DP₂ antagonist.

In some embodiments, the DP₂ antagonist is a small molecule DP₂ antagonist.

In other embodiments, the DP₂ antagonist is selected from AZD1981, ODC9101 (OC459), OC499, OC1768, OC2125, OC2184, QAV680, MLN6095, AP768, [2′-(3-Benzyl-1-ethyl-ureidomethyl)-6-methoxy-4′-trifluoromethyl-biphenyl-3-yl]-acetic acid, {3-[2-tert-Butylsulfanylmethyl-4-(2,2-dimethyl-propionylamino)-phenoxy]-4-methoxy-phenyl}-acetic acid, TM30642, TM30643, TM30089, TM27632, TM3170.

In one aspect, the detection of DP₂ receptors in the biological samples comprises contacting the biological samples with a DP₂ antibody under conditions suitable for ligand-receptor interactions.

In some embodiments, the DP₂ antibody comprises a detectable label. In one aspect, the DP₂ antibody comprises a fluorescent label.

In some embodiments, the detection of DP₂ receptor in the first biological sample and the second biological sample employs a technique selected from the group consisting of: Fluorescence-activated Cell Sorting (FACS), confocal, western blot, flow cytometry, fluorescence microscopy, quantitative polymerase chain reaction, scintillation counting, MACS.

Also provided is an in vitro diagnostic kit for detection of DP₂ receptors in a biological sample obtained from a human comprising:

(a) a DP₂ ligand; and

(b) instructions for detecting DP₂ receptors in a biological sample from a human.

In some embodiments, the DP₂ ligand is a peptide, a peptidomimetic, an antibody, an aptamer, an oligonucleotide, or a small molecule DP₂ antagonist. In some embodiments, the DP₂ ligand comprises a detectable label. In some embodiments, the DP₂ ligand comprises a fluorescent label, an enzyme label, a radioactive label, a nuclear magnetic resonance active label, a luminescent label, a chromophore label a biotin tag, or avidin tag.

In one aspect, the DP₂ ligand is a small molecule DP₂ antagonist comprising a fluorescent label, or a DP₂ antibody comprising a fluorescent label.

In some embodiments, the kit further comprises a liquid that is used to reconstitute the DP₂ ligand. In some other embodiments, the DP₂ ligand is packaged in an aqueous medium.

In some embodiments, the DP₂ ligand is bound to a solid support or coated on a solid support.

In one aspect, the biological sample is a biological fluid or biological tissue sample. In certain embodiments, the biological sample is a whole blood sample, a peripheral blood sample, a plasma sample, a sputum sample, a bronchoalveolar lavage sample, a nasal lavage sample, a tissue sample, a thin layer cytological sample, a fine needle aspirate sample, a bone marrow sample, a lymph node sample, an ascites sample, an esophageal brushing sample, a bladder wash sample, a lung wash sample, a synovial sample, a spinal fluid sample, a brain fluid sample, a ductal aspirate sample, a pleural effusion sample, or an extract or processed sample thereof.

In one aspect, the biological sample obtained from a human comprises neutrophils. In some embodiments, the biological sample comprising neutrophils is a sputum sample, a bronchoalveolar lavage sample, a nasal lavage sample, a pleural effusion sample, a fine needle aspirate sample from a lung, a lymph node sample from a lung, or a lung wash sample.

In some embodiments, the instructions are for detecting DP₂ receptors expressed on neutrophils in a biological sample from a human.

In some embodiments, detecting DP₂ receptors expressed on neutrophils comprises identifying neutrophils that express the DP₂ receptor, quantifying the number of neutrophils that express the DP₂ receptor, or identifying and quantifying the number of neutrophils that express the DP₂ receptor.

Also provided is an in vitro diagnostic kit for detection of DP₂ receptors in a biological sample obtained from a human compromising: a) a DP2 selective primary antibody; b) a fluorescent second antibody recognizing the primary antibody.

Other objects, features and advantages of the methods, compounds, compositions, kits described herein will become apparent from the following detailed description. It should be understood, however, that the detailed description and the specific examples, while indicating specific embodiments, are given by way of illustration only, since various changes and modifications within the spirit and scope of the instant disclosure will become apparent to those skilled in the art from this detailed description.

### BRIEF DESCRIPTION OF THE FIGURES

FIG. 1 presents the induction of expression of DP2 on human peripheral blood neutrophils following 4 hour incubation with LPS and/or fMLP. DP2 expression on neutrophils from human whole blood treated for 4 hours with either (A) 1 μg/ml LPS (B) 100 nM fMLP or (C) 1 μg/ml LPS plus 100 nM fMLP. In each histogram, the thin line represents DP2 staining on neutrophils from vehicle treated blood and the bold line represents DP2 staining on neutrophils from stimulated blood.

FIG. 2 presents the induction of expression of DP2 on human peripheral blood neutrophils following 22 hour incubation with LPS and/or fMLP. DP2 expression on neutrophils from human whole blood treated for 22 hours with either (A) 1 μg/ml LPS (B) 100 nM fMLP or (C) 1 μg/ml LPS plus 100 nM fMLP. In each histogram, the thin line represents DP2 staining on neutrophils from vehicle treated blood and the bold line represents DP2 staining on neutrophils from stimulated blood.

### DETAILED DESCRIPTION OF THE INVENTION

In one aspect, described herein are biomarkers that assess disease activity in inflammatory diseases or conditions. In one aspect, described herein are biomarkers that assess disease activity in inflammatory diseases or conditions, wherein the biomarkers include neutrophils. In a particular aspect, the biomarkers include neutrophils expressing the DP₂ receptor. The DP₂ receptor is not normally expressed on human neutrophils. In one aspect, neutrophils recruited to an area undergoing an inflammatory response express the DP₂ receptor. Activation of the DP₂ receptor contributes to the inflammatory process. The detection of the DP₂ receptor on neutrophils provides a biomarker that is indicative of an inflammatory process.

Biomarkers in inflammatory diseases or conditions include, but are not limited to, inflammatory cells, cytokines, cytokine antagonists, neutrophil chemoattractants, proteases, antiproteases, markers of oxidative stress, antioxidants, neutrophil products, plasma exudate markers, antimicrobial proteins, eicosanoids, eicosanoid receptors, mucins, adhesion molecules, markers of structural injury, components of signaling cascades.

Inflammatory cells include, but are not limited to, neutrophils, macrophages, lymphocytes, eosinophils, mast cells, dendritic cells, airway epithelial cells. Cytokines include, but are not limited to, IL-1β, IL-6, IL-8, IL-10, IL-17, IL-23, TNF-α, transforming growth factor β₁, IFN-γ, granulocyte-macrophage colony-stimulating factor (GM-CSF), granulocyte colony-stimulating factor (G-CSF), growth-related oncogene α, regulated on activation, normal T-Cell expressed and secreted (RANTES). Cytokine antagonists, include, but are not limited to, IL-1 receptor antagonist, soluble TNF receptor II. Neutrophil chemoattractants include, but are not limited to, IL-8, leukotriene B₄, complement-derived attractants (c5a). Proteases include, but are not limited to, elastase, matrix metalloproteinases (MMP-9), cathepsin G, proteinase 3. Antiproteases include but are not limited to, α₁-Antitrypsin, tissue inhibitors of metalloproteinases (TIMP-1), secretory leukoprotease inhibitor (SLP1), elafin, α₂-macroglobulin. Markers of oxidative stress include, but are not limited to, protein carbonyls, 3-nitrotyrosine, 3-chlorotyrosine, 8-isoprostane. Antioxidants include, but are not limited to, superoxide dismutase, glutathione, catalase, tocopherols (vitamin E), β-carotene. Other neutrophil products include, but are not limited to, DNA, myeloperoxidase, human neutrophil lipocalin. Plasma exudate markers include, but are not limited to, albumin, fibrinogen. Eicosanoids include, but are not limited to, leukotrienes, LTB₄, LTC₄, LTD₄, LTE₄, prostaglandins, prostaglandin D₂ (PGD₂), prostaglandin E₂ (PGE₂), 8-isoprostane, thromboxane B₂. Eicosanoid receptors include, but are not limited to, DP₂ receptor. Mucins include, but are not limited to, MUC5AC, MUC5B. Adhesion molecules include but are not limited to, soluble intracellular adhesion molecule. Markers of structural airway injury include, but are not limited to, elastin and collagen degradation products. Components of signaling cascades include, but are not limited to, nuclear factor-κβ, extracellular signal-regulated kinase (ERK), mitogen-activated protein kinase (MAPK), Janus kinase (JAK)/signal transducer and activator of transcription (STAT), peroxisome proliferator activating receptor (PPAR).

In one aspect, the biomarker comprises activated neutrophils. In one aspect, the biomarker comprises DP₂ receptors. In one aspect, the biomarker comprises DP₂ receptors expressed on activated neutrophils.

In one aspect, provided is a diagnostic test. In one aspect, the diagnostic test assesses whether a mammal is experiencing a disease or condition wherein neutrophilic inflammation is a component of the pathophysiology of the disease or condition. In one aspect, the detection of DP₂ receptors on neutrophils provides a diagnostic test for neutrophilic inflammation. In one aspect, the detection of DP₂ receptors on neutrophils is used to: (a) monitor disease activity; provide mechanism of action of drug candidate(s); (b) predict rapid disease progression; indicate the onset and resolution of an acute inflammatory exacerbation; (c) prescribe targeted and individualized therapies; evaluate the response to treatment with a DP₂ antagonist; (d) means of patient stratification for clinical trials involving DP₂ antagonists; and/or (e) serve as candidate surrogate endpoints in early-phase clinical trials.

### Prostaglandin D₂ (PGD₂) and DP₂

Prostaglandin D₂ (PGD₂) is an acidic lipid derived from the metabolism of arachidonic acid by cyclooxygenases and PGD₂ synthases. PGD₂ is produced by mast cells, macrophages and T_{H}2 lymphocytes in response to local tissue damage as well as in response to allergic inflammation observed in diseases such as, but not limited to, asthma, rhinitis, and atopic dermatitis.

PGD₂ is produced from the PGH₂ intermediate via hematopoietic PGD₂ synthase or lipocalin PGD₂ synthase. In the brain and central nervous system, PGD₂ is produced and thought to function in pain perception and sleep regulation. In other tissues, PGD₂ is produced primarily in immunoglobulin E (IgE) activated mast cells and to a lesser extent, in macrophages, dendritic cells, T helper 2 (T_{H}2) lymphocytes and other leukocytes. In the cell, PGD₂ is rapidly metabolized and converted to other downstream effectors including Δ¹²PGJ₂, 9α11βPGF₂, 13,14-dihydro-15-keto-PGD₂, and 15-deoxy-Δ^{12,14}PGD₂.

When PGD₂ is applied to in vivo preparations, or its overproduction is engineered by genetic manipulation the following are observed:
Vasodilatation leading to erythema (flare) and -potentiation of oedema (wheal).
Recruitment of eosinophils, neutrophils, T_{H}2 lymphocytes.
Modulation of T_{H}2-cytokine production.
Bronchoconstriction.

The secretion of PGD₂ initiates changes at the cellular level that are associated with subsequent pathophysiological effects. Thus PGD₂ provides an essential link between the early phase and late-phase allergic and/or inflammation response.

The main receptors that are activated by PGD₂ or its metabolites and mediate its effects are TP, DP₁, and CRTH2 (or DP₂). The terms CRTH2 and DP₂ refer to the same receptor and are used interchangeably herein. Likewise, another common name for DP₁ is DP, and the two terms are used interchangeably herein.

TP receptors primarily function to antagonize DP₁ receptor's effects such as promoting bronchoconstriction, vasoconstriction, and platelet aggregation. While TP receptor's main ligand is thromboxane A₂, it also binds and is activated by the PGD₂ derivative, 9α11βPGF₂. TP is a Gq-coupled prostanoid receptor that binds thromboxane with high affinity, promoting platelet aggregation and constriction of both vascular and airway smooth muscle. PGD₂ activates the TP receptor in human bronchial muscle, probably through the formation of the 11-ketoreductase metabolite 9α11βPGF2. The bronchoconstrictor effects of TP dominate over the bronchodilator effects of DP₁ in the airways.

DP₁ (or DP) is a G-protein coupled seven-transmembrane receptor that, upon activation by PGD₂ binding, leads to an increase in intracellular cAMP levels. DP₁ is expressed in the brain, bronchial smooth muscle, vascular and airway smooth muscle, dendritic cells, and platelets and induces PGD₂ dependent bronchodilation, vasodilation, platelet aggregation inhibition, and suppression of cytokine production. Genetic analysis of DP₁ function using knock-out mice has shown that mice lacking DP do not develop asthmatic responses in an ovalbumin-induced asthma model. Analysis of selective DP antagonists in guinea pig allergic rhinitis models demonstrated dramatic inhibition of early nasal responses, as assessed by sneezing, mucosal plasma exudation and eosinophil infiltration. DP antagonism alleviates allergen-induced plasma exudation in the conjunctiva in a guinea pig allergic conjunctivitis model and antigen-induced esinophil infiltration into the lung in a guinea pig asthma model.

Much of PGD₂'s pro-inflammatory activity is through interaction with DP₂ (or CRTH2). DP₂ is a G-protein coupled receptor and is typically highly expressed in T_{H}2 lymphocytes, eosinophils and basophils. Human CRTH2 is 395 amino acids in length with an estimated molecular weight of 43 kDa. Human CRTH2 exists in both an unglycosylated as well as a glycosylated protein ranging from 35-40 kDa to 50-70 kDa (Nagata et al., Prostaglandin Leukot. Essent Fatty Acids, 69, 169-177, 2003; Abe et al, Gene 227, 71-77, 1999; Nagata et al, J. Immunol. 162, 1278-1286, 1999, each of which is herein incorporated by reference). In addition to cells and tissues of hemopoietic origin human DP2 is expressed in the digestive system, heart and central nervous system. Human peripheral blood neutrophils do not normally express DP₂. It has been shown that DP₂ levels on circulating T cells correlate with the severity of atopic dermatitis. Activation of DP₂ is associated with chemotaxis and activation of TH₂ lymphocytes, eosinophils and basophils.

Despite binding PGD₂ with a similar affinity as DP₁, DP₂ is not closely structurally related to DP₁ and signals through a different mechanism—the effects of DP₂ are mediated through a Gi-dependent elevation in intracellular calcium levels and a reduction in intracellular levels of cyclic AMP. DP₂ activation is important in eosinophil recruitment in response to allergic challenge in tissues such as nasal mucosa, bronchial airways, and skin. The application of either PGD₂ or selective DP₂ agonists both exacerbate and enhance allergic responses in lung and skin. DP₂ activation triggers the recruitment of T_{H}2 lymphocytes and other leukocytes to sites of allergic inflammation and appears to have a crucial role in mediating allergic responses (such as, but not limited to, the release of chemoattractants LTB₄ and the chemokine KC, as well as an increase in neutrophilic and eosinophil infiltrate).

DP₁ and DP₂ have crucial, and complementary, roles in the physiological response of animals to PGD₂ in allergic diseases or inflammatory conditions triggered by PGD₂, such as, but not limited to, allergic rhinitis, asthma, dermatitis, and allergic conjunctivitis. The determination of the number of cells that express DP₁ or DP₂ receptors in biological samples is an attractive approach to identify the inflammatory component of allergic diseases such as, but not limited to, allergic rhinitis, asthma, dermatitis, and allergic conjunctivitis.

Neutrophils are an important source of proinflammatory cytokines and proteolytic enzymes. Neutrophils are inflammatory cells that are recruited to areas of inflammation. Neutrophilic inflammation plays a role in many inflammatory diseases or conditions. Neutrophil numbers and activation are increased in areas of inflammation. For example, neutrophil numbers and activation are increased in the airways of individuals with asthma. For example, neutrophil numbers and activation are increased in the airways of individuals during exacerbations of airway inflammation, such as, but not limited asthma. In one aspect, the asthma is severe asthma. In one aspect, the asthma is steroid-resistant asthma. Corticosteroid therapy typically does not reduce neutrophilic inflammation. As described herein, neutrophils in human peripheral blood after an inflammatory activation are shown to express the DP₂ receptor, whilst the receptor is not present on unactivated neutrophils. In one aspect, neutrophils expressing the DP₂ receptor are undergoing a switch toward cooperative functions with T cells as is noted in many inflammatory diseases or conditions, especially those that have been characterized as involving neutrophilic inflammation. In one aspect, the expression of the DP₂ receptor on neutrophils plays a role in accelerating inflammation.

Persistent activation of DP₂ on neutrophils may lead to further inflammation and tissue damage.

The expression of DP₂ on neutrophils is a biomarker that is sensitive enough to detect the status of inflammation in a mammal, and/or a change in inflammation in response to therapy for the inflammatory disease or condition. Systemic biomarkers, such as DP₂ expressed on T_{H}2 lymphocytes, eosinophils and basophils on peripheral blood leukocytes are not sensitive enough due to the fact that many inflammatory diseases or conditions are localized to specific organs or tissues. In some embodiments, neutrophils localized to areas of inflammation express DP₂.

In one aspect, presented herein are methods for identifying humans that are suitable candidates for treatment with DP₂ antagonists. In one aspect, the methods comprise detecting DP₂ receptors in a biological sample from a human. In one aspect, detecting comprises identifying cells expressing the DP₂ receptor, quantifying cells that express the DP₂ receptor, and/or identifying and quantifying cells that express the DP₂ receptor. In one aspect, the methods disclosed herein are detecting the DP₂ receptor that is expressed on neutrophils. In one aspect, the human has or had at least one symptom of an inflammatory disease or condition. In one aspect, the human has or had at least one symptom of an inflammatory disease or condition and the biological sample is from an area of the human that is involved in, associated with, or affected by the inflammation. The human is suitable to receive treatment with a DP₂ antagonist based on the detection of DP₂ receptors in the biological sample. In one aspect, the human is suitable to receive treatment with a DP₂ antagonist based on the detection of DP₂ receptors expressed on neutrophils in the biological sample.

In one aspect, the method comprises quantifying the number of cells expressing the DP₂ receptor in a biological sample from an area that is involved in, associated with, or affected by the inflammation and comparing the result to the results obtained from a biological sample from an area that is not directly involved in, associated with, or affected by the inflammation (e.g. a whole blood sample). In a non-limiting example, the number of cells expressing DP₂ receptors from a sputum sample is compared to the number of cells expressing DP₂ receptors in a whole blood sample from the same human. In one aspect, the cells are neutrophils. In one aspect, a two-fold, three fold, or more than three-fold increase in the number of cells expressing the DP₂ receptor in the biological sample from an area that is involved in, associated with, or affected by the inflammation as compared to the biological sample from an area that is not directly involved in, associated with, or affected by the inflammation indicates that the human is: suitable for therapy with a DP₂ antagonist; and/or suitable for continued therapy with a DP₂ antagonist.

In one aspect, the ratio of (1) the number of neutrophils expressing DP₂ receptors in a biological sample from an area that is involved in, associated with, or affected by the inflammation; to (2) the number of neutrophils expressing DP₂ receptors in a whole blood sample is used to classify a human as eligible to receive treatment with DP₂ receptor antagonist. The biological sample from an area that is involved in, associated with, or affected by the inflammation is, by way of example only, a sputum sample, a brochoalveolar lavage sample or a pleural effusion sample.

In one aspect, the detection of DP₂ receptors in a biological sample from an area that is involved in, associated with, or affected by the inflammation provides information that is used to diagnose the inflammatory disease or condition as a PGD₂-dependent or PGD₂-mediated disease or condition. In one aspect, the detection of DP₂ receptors expressed on neutrophils in a biological sample from an area that is involved in, associated with, or affected by the inflammation is used to diagnose the inflammatory disease or condition as a PGD₂-dependent or PGD₂-mediated disease or condition.

In one aspect, the detection of DP₂ receptors expressed on neutrophils in a biological sample from an area that is involved in, associated with, or affected by the inflammation is used to diagnose the inflammatory disease or condition as involving neutrophilic inflammation. In some cases, the neutrophilic aspect of certain inflammatory conditions are difficult to treat with conventional treatment modalities, such as, but not limited to, corticosteroids. In these cases, many of the symptoms of the inflammatory disease or condition are not addressed by conventional treatment modalities. In one aspect, the detection of DP₂ receptors expressed on neutrophils in a biological sample from an area that is involved in, associated with, or affected by the inflammation provides the information that determines the suitability of treating the human with a DP₂ antagonist. In one aspect, the detection of DP₂ receptors expressed on neutrophils provides the information that is used to determine whether or not the human is suitable to receive combination therapy with two or more therapeutic agents, where one of the therapeutic agents is a DP₂ antagonist.

In some embodiments, when the PGD₂-dependent or PGD₂-mediated condition or disease is insensitive to treatment with steroids, and the detection of DP₂ receptors expressed on neutrophils in the biological sample provides information that is used to determine the suitability of the human for therapy with a DP₂ antagonist (either mono-therapy or combination therapy).

In one aspect, the methods of detecting DP₂ receptors disclosed herein allow for the monitoring of the status of an inflammatory disease or condition. In a particular aspect of this embodiment, the detection of DP₂ receptors expressed on neutrophils (the number of neutrophils expressing the DP₂ receptor and/or the expression level of the DP₂ receptor on neutrophils) in a biological sample from the human provides information that is used to determine if the human is in need of therapy with a DP₂ antagonist or is in continued need of therapy with a DP₂ antagonist.

The methods disclosed herein allow for the determination of therapeutically effective amounts of DP₂ antagonists that are administered to humans, or the adjustment of the therapeutically effective amounts of the DP₂ antagonists that are administered to the human during chronic administration over an extended period of time, including throughout the duration of the human's life, in order to ameliorate or otherwise control or limit the symptoms of the inflammatory disease or condition.

The method disclosed herein also provide information that is used to determine if a human is at risk for experiencing at least one symptom of a PGD₂-dependent or PGD₂-mediated disease or condition. In such methods, a biological sample from the human is contacted with at least one endotoxin. The detection of DP₂ receptors expressed on neutrophils in the stimulated biological sample is performed. The degree by which DP₂ receptors are detected in the stimulated sample (expression level of the DP₂ receptor on neutrophils and the number of neutrophils expressing the DP₂ receptor) when compared to an unstimulated sample provides information regarding the likelihood that the human will be at risk for experiencing at least one symptom of a PGD₂-dependent or PGD₂-mediated disease or condition. Such information also provides an indication of the severity of the symptoms that will be experienced. If the human is already experiencing at least one symptom of an inflammatory disease or condition, such information ascertains the likelihood that the symptoms will get worse. The human may be in a period of remission of an inflammatory disease or condition (i.e. symptom-free) or may not yet have experienced any symptoms of an inflammatory disease or condition.

The methods disclosed herein further provide for the determination of optimized dose levels of DP₂ antagonists for humans in need thereof. In certain embodiments, the methods disclosed herein provide early diagnoses of PGD₂ dependent diseases or conditions and allow for the prophylactic administration of DP₂ antagonists. When the PGD₂-dependent or PGD₂-mediated condition or disease is insensitive to treatment with steroids, the methods disclosed herein allow for therapeutic intervention via administration of a DP₂ antagonist. In other embodiments, the methods disclosed herein identify humans already suffering from a PGD₂-dependent or PGD₂-mediated disease or condition.

The detection of DP₂ receptors in a biological sample from a human provides information regarding the clinical efficacy of a DP₂ antagonist. Comparison of the results from detecting DP2 receptors (expression level and/or cell numbers) from a biological sample prior to the administration of a DP₂ antagonist to the results from detecting DP₂ receptors (expression level and/or cell numbers) from a biological sample after the administration of a DP₂ antagonist provides information regarding the clinical efficacy of the DP2 antagonist. In another aspect, such comparison provides information relevant to the determination of a therapeutically effective amount of the DP₂ antagonist.

### Diseases or Conditions

In one aspect, assays described herein are used to diagnose diseases or conditions as PGD₂-dependent or PGD₂-mediated. The term “PGD₂-dependent”, as used herein, refers to conditions or disorders that would not occur, or would not occur to the same extent, in the absence of PGD₂. The term “PGD₂-mediated”, as used herein, refers to refers to conditions or disorders that might occur in the absence of PGD₂ but can occur in the presence of PGD₂.

In one aspect, PGD₂-dependent or PGD₂-mediated diseases or conditions include, but are not limited to, asthma, rhinitis, allergic conjunctivitis, atopic dermatitis, chronic obstructive pulmonary disease (COPD), pulmonary hypertension, interstitial lung fibrosis, cystic fibrosis, arthritis, allergy, psoriasis, inflammatory bowel disease, adult respiratory distress syndrome, myocardial infarction, stroke, arthritis, wound healing, endotoxic shock, cancer, pain, eosinophilic esophagitis, eosinophil-associated gastrointestinal disorders (EGID), idiopathic hypereosinophilic syndrome, otitis, airway constriction, mucus secretion, nasal congestion, increased microvascular permeability and recruitment of eosinophils, urticaria, sinusitis, uveitis, angioedema, anaphylaxia, chronic cough and Churg Strauss syndrome.

In one aspect, assays described herein are used to diagnose individuals with neutrophilic inflammation. Neutrophilic inflammation is involved in many inflammatory diseases or conditions. Neutrophilic inflammation is involved in many inflammatory diseases or conditions, such as respiratory diseases or conditions or allergic diseases or conditions.

In one aspect, assays described herein diagnose individuals as suitable candidates for therapy with DP₂ antagonist compounds. In one aspect, the individuals include those individuals with an inflammatory disease or condition. In one aspect, the inflammatory disease or condition is a respiratory disease or condition. In another aspect, the inflammatory disease or condition is an allergic disease or condition.

The term “inflammatory disease or condition” refers to those diseases or conditions that are characterized by one or more of the signs of pain, heat, redness, swelling, and loss of function (temporary or permanent). In one aspect, an inflammatory disease or condition is characterized by the recruitment of neutrophils to the area involved in inflammation. In one aspect, the inflammatory disease or condition is triggered by PGD₂.

Inflammation takes many forms and includes, but is not limited to, inflammation that is characterized by one or more of the following: acute, adhesive, atrophic, catarrhal, chronic, cirrhotic, diffuse, disseminated, exudative, fibrinous, fibrosing, focal, granulomatous, hyperplastic, hypertrophic, interstitial, metastatic, necrotic, obliterative, parenchymatous, plastic, productive, proliferous, pseudomembranous, purulent, sclerosing, seroplastic, serous, simple, specific, subacute, suppurative, toxic, traumatic, and/or ulcerative.

Inflammatory disorders further include, without being limited to those affecting the blood vessels (polyarteritis, temporal arteritis); joints (arthritis: crystalline, osteo-, psoriatic, reactive, rheumatoid, Reiter's); gastrointestinal tract (colitis); skin (dermatitis); organs (lungs, liver, pancreas); or multiple organs and tissues (systemic lupus erythematosus).

Inflammatory diseases or conditions include, but are not limited to, respiratory diseases or conditions, allergic diseases or conditions, psoriasis, inflammatory bowel disease, adult respiratory distress syndrome, myocardial infarction, congestive heart failure, stroke, arthritis, wound healing, endotoxic shock, cancer, pain, eosinophilic esophagitis, eosinophil-associated gastrointestinal disorders (EGID), idiopathic hypereosinophilic syndrome, otitis, airway constriction, mucus secretion, nasal congestion, increased microvascular permeability and recruitment of eosinophils, urticaria, sinusitis, uveitis, angioedema, anaphylaxia, chronic cough, Churg Strauss syndrome, rheumatoid arthritis, ankylosing spondylitis, osteoarthritis, lupus, graft versus host disease, tissue transplant rejection, ischemic conditions, epilepsy, Alzheimer's disease, Parkinson's disease, vitiligo, Wegener's granulomatosis, gout, eczema, dermatitis, coronary infarct damage, chronic inflammation, smooth muscle proliferation disorders, multiple sclerosis, and acute leukocyte-mediated lung injury. In some embodiments, inflammatory conditions are immune or anaphylactic disorders associated with infiltration of leukocytes into inflamed tissues or organs. In other embodiments, inflammatory conditions are associated with T-lymphocyte activation.

Allergic diseases or conditions include, but are not limited to, ocular inflammation and allergic conjunctivitis, vernal keratoconjunctivitis, papillary conjunctivitis, allergic rhinitis, asthma, dermatitis.

In one aspect, the inflammatory disease or condition is an ocular disease or condition. The term “ocular disease or condition,” as used herein, refers to diseases or conditions which affect the eye or eyes and potentially the surrounding tissues as well. Ocular diseases or conditions include, but are not limited to, conjunctivitis, retinitis, scleritis, uveitis, allergic conjunctivitis, vernal conjunctivitis, papillary conjunctivitis.

The term “respiratory disease or condition,” as used herein, refers to diseases or conditions affecting the organs that are involved in breathing, such as the nose, throat, larynx, eustachian tubes, trachea, bronchi, lungs, pulmonary tissues, related muscles (e.g., diaphragm and intercostals), sacs (e.g., alveoli) and nerves.

Respiratory diseases or conditions include, but are not limited to, asthma, adult respiratory distress syndrome and allergic (extrinsic) asthma, non-allergic (intrinsic) asthma, acute severe asthma, chronic asthma, clinical asthma, nocturnal asthma, allergen-induced asthma, aspirin-sensitive asthma, exercise-induced asthma, isocapnic hyperventilation, child-onset asthma, adult-onset asthma, cough-variant asthma, occupational asthma, steroid-resistant asthma, seasonal asthma, seasonal allergic rhinitis, perennial allergic rhinitis, chronic obstructive pulmonary disease, including chronic bronchitis or emphysema, pulmonary hypertension, interstitial lung fibrosis and/or airway inflammation and cystic fibrosis, and hypoxia.

In one aspect, the respiratory disease or condition is asthma. The term “asthma” as used herein refers to any disorder of the lungs characterized by variations in pulmonary gas flow associated with airway constriction of whatever cause (intrinsic, extrinsic, or both; allergic or non-allergic). The term asthma may be used with one or more adjectives to indicate cause. In one aspect, the type of asthma is allergic (extrinsic) asthma, non-allergic (intrinsic) asthma, acute severe asthma, chronic asthma, clinical asthma, nocturnal asthma, allergen-induced asthma, aspirin-sensitive asthma, exercise-induced asthma, child-onset asthma, adult-onset asthma, cough-variant asthma, occupational asthma, steroid-resistant asthma, or seasonal asthma.

The term “rhinitis” as used herein refers to any disorder of the nose in which there is inflammation of the mucous lining of the nose by whatever cause (intrinsic, extrinsic or both; allergic or non-allergic).

The term “iatrogenic” means a PGD₂-dependent or PGD₂-mediated condition, disorder, or disease created or worsened by medical or surgical therapy.

In one aspect, the respiratory disease or condition includes, but is not limited to, adult respiratory distress syndrome and allergic (extrinsic) asthma, non-allergic (intrinsic) asthma, acute severe asthma, chronic asthma, clinical asthma, nocturnal asthma, allergen-induced asthma, aspirin-sensitive asthma, exercise-induced asthma, isocapnic hyperventilation, child-onset asthma, adult-onset asthma, cough-variant asthma, occupational asthma, steroid-resistant asthma, seasonal asthma.

In one aspect, the allergic disease or condition is rhinitis. In a further embodiment of this aspect, the allergic disease or condition includes, but is not limited to, allergic (extrinsic) rhinitis, non-allergic (intrinsic) rhinitis, chronic rhinitis, allergen-induced rhinitis, aspirin-sensitive rhinitis, child-onset rhinitis, adult-onset rhinitis, occupational rhinitis, steroid-resistant rhinitis, seasonal rhinitis, perennial rhinitis, rhinosinusitis, and rhinopolyposis.

In another aspect, the inflammatory disease or condition is chronic obstructive pulmonary disease. In a further embodiment of this aspect, chronic obstructive pulmonary disease includes, but is not limited to, chronic bronchitis and/or emphysema, pulmonary hypertension, interstitial lung fibrosis and/or airway inflammation and cystic fibrosis.

### DP₂ Antagonists

In one aspect, the assays described herein determine the eligibility of an individual for therapy with DP₂ antagonist, assess the effectiveness of therapy with a DP₂ antagonist, determine a therapeutically effective amount of a DP₂ antagonist for treating an inflammatory disease or condition, determine if add-on therapy with a DP₂ antagonist is appropriate, determine if therapy with a DP₂ antagonist is appropriate, among other uses. In one aspect, the DP₂ antagonist is a small molecule DP₂ antagonist.

Examples of small molecule DP₂ antagonists include, and are not limited to, compounds disclosed in U.S. provisional application No. 61/031,310 (entitled “Antagonists of Prostaglandin D₂ receptors”); International patent application no. PCT/US09/35174 (entitled “Antagonists of Prostaglandin D₂ receptors”); U.S. provisional application No. 60/985,919 (entitled “Antagonists of PGD₂ receptors”); International patent application no. PCT/US08/82056 (entitled “Antagonists of PGD₂ receptors”); U.S. provisional application No. 60/985,913 (entitled “Antagonists of PGD₂ receptors”); International patent application no. PCT/US08/82082 (entitled “Antagonists of PGD2 receptors”); U.S. provisional application No. 61/025,597 (entitled “N,N-disubstituted aminoalkylbiphenyl antagonists of prostaglandin D₂ receptors”); International patent application no. PCT/US09/32495 (entitled “N,N-disubstituted aminoalkylbiphenyl antagonists of prostaglandin D₂ receptors”); International patent application no. PCT/US09/32499 (entitled “N,N-disubstituted aminoalkylbiphenyl antagonists of prostaglandin D₂ receptors”); U.S. application Ser. No. 12/362,439 (entitled “N,N-disubstituted aminoalkylbiphenyl antagonists of prostaglandin D₂ receptors”); U.S. provisional application No. 61/110,496 (entitled “N,N-disubstituted aminoalkylbiphenyl antagonists of prostaglandin D₂ receptors”); U.S. provisional application No. 61/028,804 (entitled “Cyclic diaryl ether compounds as antagonists of prostaglandin D₂ receptors”); International patent application no. PCT/US09/33961 (entitled “Cyclic diaryl ether compounds as antagonists of prostaglandin D₂ receptors”); U.S. provisional application No. 61/041,869 (entitled “Aminoalkylphenyl antagonists of prostaglandin D₂ receptors”); International patent application no. PCT/US09/38291 (entitled “Aminoalkylphenyl antagonists of prostaglandin D₂ receptors”); U.S. provisional application No. 61/078,311 (entitled “Heteroalkyl antagonists of prostaglandin D₂ receptors”); International patent application no. PCT/US09/49621 (entitled “Antagonists of prostaglandin D₂ receptors”); International patent application no. PCT/US09/49631 (entitled “Antagonists of prostaglandin D₂ receptors”); U.S. application Ser. No. 12/497,343 (entitled “Antagonists of prostaglandin D₂ receptors”); U.S. provisional application No. 61/101,074 (entitled “Heteroaryl antagonists of prostaglandin D₂ receptors”); International patent application no. PCT/US09/58655 (entitled “Heteroaryl antagonists of prostaglandin D₂ receptors”); International patent application no. PCT/US09/58663 (entitled “Heteroaryl antagonists of prostaglandin D₂ receptors”); Ser. No. 12/568,571 (entitled “Heteroaryl antagonists of prostaglandin D2 receptors”); U.S. provisional application No. 61/054,093 (entitled “Tricyclic antagonists of prostaglandin D₂ receptors”); U.S. provisional application No. 61/107,638 (entitled “Tricyclic antagonists of prostaglandin D₂ receptors”); International patent application no. PCT/US09/44219 (entitled “Tricyclic antagonists of prostaglandin D₂ receptors”); U.S. provisional application No. 61/075,242 (entitled “Cycloaklane[B]indole antagonists of prostaglandin D₂ receptors”); International patent application no. PCT/US09/48327 (entitled “Cycloaklane[B]indole antagonists of prostaglandin D₂ receptors”); U.S. provisional application No. 61/101,964 (entitled “Heteroaryl antagonists of prostaglandin D₂ receptors”); International patent application no. PCT/US09/59256 (entitled “Heteroaryl antagonists of prostaglandin D₂ receptors”); U.S. provisional application No. 61/103,872 (entitled “Heteroalkyl biphenyl antagonists of prostaglandin D₂ receptors”); International patent application no. PCT/US09/59891 (entitled “Heteroalkyl biphenyl antagonists of prostaglandin D₂ receptors”); U.S. provisional application No. 61/115,259 (entitled “Heterocyclic antagonists of prostaglandin D₂ receptors”); International patent application no. PCT/US09/64630 (entitled “Heterocyclic antagonists of prostaglandin D₂ receptors”); U.S. provisional application No. 61/112,044 (entitled “Cycloaklane[B]azaindole antagonists of prostaglandin D₂ receptors”) International patent application no. PCT/US09/63439 (entitled “Cycloaklane[B]azaindole antagonists of prostaglandin D₂ receptors”); International patent application no. PCT/US09/63438 (entitled “Cycloaklane[B]azaindole antagonists of prostaglandin D₂ receptors”); U.S. application Ser. No. 12/613,424 (entitled “Cycloaklane[B]azaindole antagonists of prostaglandin D₂ receptors”); each of which is herein incorporated by reference in their entirety.

In one aspect, the small molecule DP₂ antagonist is selected from: Ramatroban (Baynas, BAY u3405), AZD1981, ODC9101 (OC459), OC499, OC1768, OC2125, OC2184, QAV680, MLN6095, AP768, [2′-(3-Benzyl-1-ethyl-ureidomethyl)-6-methoxy-4′-trifluoromethyl-biphenyl-3-yl]-acetic acid, {3-[2-tert-Butylsulfanylmethyl-4-(2,2-dimethyl-propionylamino)-phenoxy]-4-methoxy-phenyl}-acetic acid.

Other small molecule DP₂ antagonists include, and are not limited to, ramatroban, TM30642, TM30643, TM30089, TM27632, TM3170, candesartan, as well as compounds disclosed in Pettipher et al, Nature Reviews Drug Discovery, vol. 6, 313-325, 2007; Medina et al., Annual Reports in Medicinal Chemistry, Vol. 41, 221-235, 2006, each of which is herein incorporated by reference).

In some embodiments, the DP₂ anatgonists are adminstered to humans as pharmaceutically acceptable salts. In some embodiments, pharmaceutically acceptable salts are obtained by reacting a DP₂ antagonist with acids. Pharmaceutically acceptable salts are also obtained by reacting a DP₂ antagonist with a base. DP₂ antagonists may be formed as, and/or used as, pharmaceutically acceptable salts. The type of pharmaceutical acceptable salts, include, but are not limited to: (1) acid addition salts, formed by reacting the free base form of the compound with a pharmaceutically acceptable inorganic acid, such as, for example, hydrochloric acid, hydrobromic acid, sulfuric acid, phosphoric acid, metaphosphoric acid, and the like; or with an organic acid, such as, for example, acetic acid, propionic acid, hexanoic acid, cyclopentanepropionic acid, glycolic acid, pyruvic acid, lactic acid, malonic acid, succinic acid, malic acid, maleic acid, fumaric acid, trifluoroacetic acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, toluenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, butyric acid, phenylacetic acid, phenylbutyric acid, valproic acid, and the like; (2) salts formed when an acidic proton present in the parent compound is replaced by a metal ion, e.g., an alkali metal ion (e.g. lithium, sodium, potassium), an alkaline earth ion (e.g. magnesium, or calcium), or an aluminum ion. In some cases, DP₂ anatgonists are reacted with an organic base, such as, but not limited to, ethanolamine, diethanolamine, triethanolamine, tromethamine, N-methylglucamine, dicyclohexylamine, tris(hydroxymethyl)methylamine. In other cases, DP₂ anatgonists form salts with amino acids such as, but not limited to, arginine, lysine, and the like. Acceptable inorganic bases used to form salts with compounds that include an acidic proton, include, but are not limited to, aluminum hydroxide, calcium hydroxide, potassium hydroxide, sodium carbonate, sodium hydroxide, and the like.

The DP₂ anatgonists are included in the formulations described herein as pharmaceutically acceptable salts, and/or pharmaceutically acceptable solvates. In one aspect, DP₂ anatgonists are included in the formulations described herein in free acid form or free base form.

In some embodiments, the DP₂ antagonists possess one or more stereocenters and each center exists independently in either the R or S configuration. The compounds presented herein include all diastereomeric, enantiomeric, and epimeric forms as well as the appropriate mixtures thereof.

In one aspect, the DP₂ antagonist is formulated for intravenous injection, subcutaneous injection, oral administration, inhalation, nasal administration, topical administration, ophthalmic administration or otic administration. In some embodiments, the pharmaceutical composition is a tablet, a pill, a capsule, a liquid, an inhalant, a nasal spray solution, a suppository, a suspension, a gel, a colloid, a dispersion, a suspension, a solution, an emulsion, an ointment, a lotion, an eye drop or an ear drop.

Pharmaceutical formulations that comprise the DP₂ antagonists are administerable in a variety of ways by multiple administration routes, including but not limited to, oral, parenteral (e.g., intravenous, subcutaneous, intramuscular), intranasal, buccal, topical or transdermal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

In some embodiments, the DP₂ antagonists are administered orally.

In some embodiments, the DP₂ antagonists are administered topically. In such embodiments, the DP₂ antagonist is formulated into a variety of topically administrable compositions, such as solutions, suspensions, lotions, gels, pastes, shampoos, scrubs, rubs, smears, medicated sticks, medicated bandages, balms, creams or ointments. Such pharmaceutical compounds can contain solubilizers, stabilizers, tonicity enhancing agents, buffers and preservatives.

In another aspect, the DP₂ antagonists are administered by intranasal administration.

In another aspect, the DP₂ antagonists are formulated for intranasal administration. Such formulations include nasal sprays, nasal mists, and the like.

In another aspect, the DP₂ antagonists are formulated as eye drops.

In one aspect, the DP₂ antagonists are administered topically to the skin.

In one aspect, the DP₂ antagonist: (a) is systemically administered to the mammal; and/or (b) is administered orally to the mammal; and/or (c) is intravenously administered to the mammal; and/or (d) is administered by inhalation; and/or (e) is administered by nasal administration; or and/or (f) is administered by injection to the mammal; and/or (g) is administered topically (dermal) to the mammal; and/or (h) is administered by ophthalmic administration; and/or (i) is administered rectally to the mammal.

### DEFINITIONS

The term “modulate,” as used herein, means to interact with a target either directly or indirectly so as to alter the activity of the target, including, by way of example only, to enhance the activity of the target, to inhibit the activity of the target, to limit the activity of the target, or to extend the activity of the target.

The term “modulator” as used herein, refers to a molecule that interacts with a target either directly or indirectly. The interactions include, but are not limited to, the interactions of an agonist, partial agonist, an inverse agonist and antagonist. In one embodiment, a DP₂ modulator is a DP₂ antagonist.

The term “ligand,” as used herein refers to a molecule that binds with a target. The binding of the ligand with the target may occur at the active site of the target or at an allosteric site. The binding may be reversible or irreversible, and may be covalent or non-covalent.

The term “detectable label” refers to any reporter group that is attached to a ligand and does not interfere with the binding properties of the ligand.

The term “biological sample” refers to any bodily fluid or tissue that is obtained from a human.

The term “transformed” when applied to a biological sample refers to a biological sample that has undergone at least one manipulation to allow the detection of a desired signal by an analytical instrument so as to provide information about a detectable component in the biological sample. The term applies whether or not the signal is detected provided that the sample has been manipulated to allow detection if the detectable component (e.g., if the untransformed biological sample has no detectable components, then the aforementioned manipulations will result in no detected signal by the analytical instrument; this information, however, is still useful). Non-limiting methods of providing a transformed biological sample are provided herein.

The term “agonist,” as used herein, refers to a molecule such as a compound, a drug, an enzyme activator or a hormone modulator that binds to a specific receptor and triggers a response in the cell. An agonist mimics the action of an endogenous ligand (such as a prostaglandin or hormone) that binds to the same receptor.

The term “antagonist,” as used herein, refers to a molecule such as a compound, which diminishes, inhibits, or prevents the action of another molecule or the activity of a receptor site. Antagonists include, but are not limited to, competitive antagonists, non-competitive antagonists, uncompetitive antagonists, partial agonists and inverse agonists.

Competitive antagonists reversibly bind to receptors at the same binding site (active site) as the endogenous ligand or agonist, but without activating the receptor.

Non-competitive antagonists (also known as allosteric antagonists) bind to a distinctly separate binding site from the agonist, exerting their action to that receptor via the other binding site. Non-competitive antagonists do not compete with agonists for binding. The bound antagonists may result in a decreased affinity of an agonist for that receptor, or alternatively may prevent conformational changes in the receptor required for receptor activation after the agonist binds.

Uncompetitive antagonists differ from non-competitive antagonists in that they require receptor activation by an agonist before they can bind to a separate allosteric binding site.

Partial agonists are defined as drugs which, at a given receptor, might differ in the amplitude of the functional response that they elicit after maximal receptor occupancy. Although they are agonists, partial agonists can act as a competitive antagonist if co-administered with a full agonist, as it competes with the full agonist for receptor occupancy and producing a net decrease in the receptor activation observed with the full agonist alone.

An inverse agonist can have effects similar to an antagonist, but causes a distinct set of downstream biological responses. Constitutively active receptors which exhibit intrinsic or basal activity can have inverse agonists, which not only block the effects of binding agonists like a classical antagonist, but inhibit the basal activity of the receptor.

The terms “effective amount” or “therapeutically effective amount,” as used herein, refer to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case may be determined using techniques, such as a dose escalation study.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

The terms “kit” and “article of manufacture” are used as synonyms.

The term “subject”, “individual” or “mammal” encompasses humans and non-humans. In one embodiment, the “subject”, “individual” or “mammal” is a human.

The terms “treat,” “treating” or “treatment,” as used herein, include alleviating, abating or ameliorating a disease or condition symptoms, preventing additional symptoms, ameliorating or preventing the underlying causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

### Biological Samples

In one aspect, the biological samples that are assayed comprise biological tissues and biological fluids. In one aspect, the biological samples are assayed for neutrophils expressing the DP₂ receptors. In one aspect, the biological samples that are assayed for neutrophils expressing the DP₂ receptor include a whole blood sample, a peripheral blood sample, a plasma sample, a sputum sample, a saliva sample, a bronchoalveolar lavage sample, a nasal lavage sample, a tear sample, a tissue sample, a thin layer cytological sample, a fine needle aspirate sample, a bone marrow sample, a lymph node sample, a synovial sample, an ascites sample, an esophageal brushing sample, a bladder wash sample, a lung wash sample, a spinal fluid sample, a brain fluid sample, a ductal aspirate sample, a pleural effusion sample, or an extract or processed sample thereof.

In one aspect, the biological sample is a sputum sample or a bronchoalveolar lavage sample. In one aspect, the biological sample is a sputum sample. In one aspect, the biological sample is a bronchoalveolar lavage sample.

In some embodiments, the biological sample is obtained from or around an organ or tissue. In one aspect, the biological sample is obtained from or around the lungs. In one aspect, the biological sample is a whole blood sample.

In one aspect, the biological sample is obtained from an area of the human that is undergoing inflammation. In one aspect, the biological sample is used to determine the amount of localized inflammation. In one aspect, the biological sample is a whole blood sample. In one aspect, two biological samples are assayed, one from an area of inflammation, and one remote from the area of the area of inflammation. In one aspect, a biological sample obtained from or around the lungs is assayed and the results are compared to assay results using a biological sample remote from the area of inflammation, such as, but not limited to a whole blood sample.

In some embodiments, the biological samples are used directly in the assays described herein. In other embodiments, the samples are contacted with an endotoxin. Endotoxins include, but are not limited to, lipopolysaccharide (LPS) and lipooligosaccharide (LOS). In other embodiments, the samples are contacted with neutrophil chemoattractants/activating ligands. In one aspect, neutrophil chemoattractants/activating ligands include ligands that bind to neutrophil receptors and mediate chemotaxis and activation of neutrophils. Neutrophil chemoattractants/activating ligands include, but are not limited to, peptide ligands such as formyl-methionine-leucine-phenylalanine (fMLP).

In other embodiments, the samples are contacted with both endotoxins and neutrophil chemoattractants/activating ligands. In one aspect, LPS enhances the effects of neutrophil chemoattractants/activating ligands in the biological samples.

In further embodiments, the samples are processed prior to assaying for DP₂ receptors. Processing steps include any of the following: extraction of the biological sample with a suitable solvent, precipitation of extracellular debris or other processing steps that are known.

In one aspect, neutrophil chemoattractants/activating ligands are selected from ligands that bind to chemotaxis receptors. In one aspect, neutrophil chemoattractants/activating ligands are selected from ligands that bind to chemotaxis receptors present on neutrophils. In one aspect, neutrophil chemoattractants/activating ligands are selected from ligands that mediate chemotaxis and activation of neutrophils.

Chemotaxis receptors include, but are not limited to, formyl peptide receptors (FPR), chemokine receptors (CCR or CXCR) and leukotriene receptors. Examples of formyl peptide receptor (FPR) ligand include, but are not limited to formyl peptides. In one aspect, formyl peptides are di-, tri-, tetrapeptides of bacterial origin. In one aspect, formyl peptides are released from bacteria in vivo or after decomposition of the cell. In one aspect, a formyl peptide is N-formylmethionyl-leucyl-phenylalanine (fMLF or fMLP).

Chemokines bind to chemokine receptors. Leukotrienes bind to leukotriene receptors. Complements, such as complement 3a (C3a) and complement 5a (C5a) are contemplated.

### Detection of DP₂ Receptors

In one aspect, the detection of DP₂ receptors comprises transforming DP₂ receptors into DP₂ receptors that are detectable. In one aspect, DP₂ receptors that are detectable comprise complexes of DP₂ ligands interacting with the DP₂ receptor, where the DP₂ ligand comprises a detectable label. In one aspect, transforming DP₂ receptors into DP₂ receptors that are detectable comprises contacting DP₂ receptors with a DP₂ ligand under conditions that are suitable for ligand-receptor interactions in order to form a ligand-receptor complex, wherein DP₂ ligand comprises a detectable label and the ligand-receptor complexes are detectable with at least one analytical instrument or device.

In one aspect, the detection of DP₂ receptors in a biological sample comprises contacting the biological sample with a DP₂ ligand comprising a detectable label. In one aspect, the DP₂ ligand is a peptide, a peptidomimetic, an antibody, an aptamer, an oligonucleotide or a small molecule DP₂ modulator. The detectable label is any group or moiety that is identifiable with at least one analytical instrument. In some aspects, the detectable label is any group or moiety that is identifiable upon visual inspection by at least one human. Labels (or tags) include, but are not limited to, any of the groups or moieties disclosed herein or known in the art of identifying protein receptors. In one aspect, labels (or tags) include, but are not limited to, fluorescent label(s), enzyme label(s), radioactive label(s), nuclear magnetic resonance active label(s), paramagnetic resonance label(s), luminescent label(s), chromophore label(s), biotin tag(s), avidin tag(s). The assay used to detect DP₂ receptors in biological samples depends on the type of tag attached to the DP₂ ligand. By way of example, when the tag is a fluorescent label, methods known in the art, including, by way of example, the FACS technique, are used for identifying and counting the number of cells expressing DP₂ receptors, see Current Protocols in Cytometry, 2007 by John Wiley and Sons, Inc., chapters 1, 2, 5, 10, 12 and appendices, and Current Protocols in Immunology, 2007 by John Wiley and Sons, Inc., chapter 5, which are incorporated by reference for such disclosure. In another instance, when the tag is a radiolabel, a gamma counter is used for detection of expression levels of DP₂ receptors. In yet another instance, when the tag is biotin, an avidin-based colormetric assay is used for the detection of expression levels of DP₂ receptors. The biotin-avidin assay may be an enzyme linked immunosorbent assay (ELISA) or a solid phase radioimmunosorbent assay (SPRIA). Biotin-avidin detection systems known in the art, include a double-antibody sandwich ELISA (M-Ab ELISA) which uses a biotinylated second antibody and an avidin-alkaline phosphatase detection system, see e.g., Diaco, J Gen Virol 1985, 66, 2089-2094; and an immunoradiometric assay which uses a I¹²⁵-labeled antibody coupled to a biotin-avidin bridge with avidin bound to polystyrene beads, see, Zahradnik, Clin. Chem. 1989, 35, 804-807, which are incorporated by reference for such disclosure.

In one aspect, the detection of DP2 receptor protein is performed using quantititative polymerase chain reaction for DP2 messenger RNA. Messenger RNA (mRNA) levels of DP2 may by analyzed using assays such as, for example, quantititative polymerase chain reaction, reverse transcriptase-polymerase chain reaction (RT-PCR), Northern hybridization, in situ hybridization and quantitative RT-PCR (qRT-PCR).

In some embodiments, protein levels of DP2 are analyzed using assays such as, for example, an enzyme linked immunosorbent assay (ELISA), a Western blot, immunohistochemistry, immunoprecipitation, immunofluorescence, enzyme immunoassay (EIA) and radioimmunoassay (RIA).

In one aspect, biological samples from humans will undergo processing for example with selective antibody detection of DP2 and quantitation of neutrophil expression of DP2. In one aspect, the cells are stained with an anti-CD16 antibody conjugated to the fluorochrome PC5 (Beckman Coulter, clone 3G8) and an anti-DP2 antibody conjugated to the fluorochrome R-phycoerytherin (PE) (Miltenyi Biotech, clone BM16) for 30 minutes on ice. The cells are washed twice with cold PBS and resuspended in cold cytofix (BD Biosciences). DP2 expression on the neutrophils is analyzed using a FACSCalibur (BD Biosciences) and gating on the CD16 high, side scatter (SSC) high cells.

### DP₂ Ligands

DP₂ ligands include, but are not limited to, prostaglandins, peptides, polypeptides, peptidomimetic compounds, proteins, including antibodies and antibody ligand binding domains, hormones, oligonucleotides, nucleic acids such as DNA or RNA, aptamers, and small molecule compounds.

In one aspect, the DP₂ ligand is a DP₂ antibody specific for the DP₂ receptor. The antibody is tagged with an appropriate label depending on the technique to be used for the detection of DP₂ receptors. By way of non-limiting examples, DP₂ antibodies that are compatible with the methods disclosed herein include anti CRTH2 antibody BM7 (Nagata, K. et al., J. Immunol., 162: 1278-1286, 1999; Nagata, K., et al., FEBS Lett., 459: 195-199, 1999; U.S. Pat. No. 6,884,593, which are incorporated herein by reference). Examples of two labelled DP₂ ligands include CRTH2 antibodies (clone BM16 (isotype: rat IgG2a) conjugated to R-phycoerythrin (PE) or to biotin (see Miltenyi Biotec, catalog no. 130-091-238 and 130-091-239).

Fragments of antibodies are also contemplated. In one aspect, the DP₂ ligand is an antigen binding fragment of a DP₂ antibody (e.g. Fab, F(ab′)₂, Fv, scFv, single binding chain polypeptide).

In one aspect, the antibody is a polyclonal, monoclonal or chimeric antibody or a fragment thereof. Polyclonal antibodies are heterogeneous populations of antibody molecules derived from the sera of animals immunized with an antigen. A monoclonal antibody (MAb) contains a substantially homogeneous population of antibodies specific to antigens, which population contains substantially similar epitope binding sites. MAbs are obtained by methods known to those skilled in the art. See, for example Kohler and Milstein, Nature 256:495-497 (1975); U.S. Pat. No. 4,376,110; Ausubel et al., eds., Current Protocols in Molecular Biology, Greene Publishing Assoc. and Wiley Interscience, N.Y., (1987, 1992); and Harlow and Lane ANTIBODIES: A Laboratory Manual Cold Spring Harbor Laboratory (1988); Colligan et al., eds., Current Protocols in Immunology, Greene Publishing Assoc. and Wiley Interscience, N.Y., (1992, 1993), the contents of which references are incorporated herein by reference for such disclosures.

Chimeric antibodies are molecules wherein different portions of the molecules are derived from different animal species. Chimeric antibodies and methods for their production are known in the art (Cabilly et al., Proc. Natl. Acad. Sci. USA 81:3273-3277 (1984); Morrison et al., Proc. Natl. Acad. Sci. USA 81:6851-6855 (1984); Boulianne et al., Nature 312:643-646 (1984); Cabilly et al., European Patent Application 125023; Neuberger et al., Nature 314:268-270 (1985); Taniguchi et al., European Patent Application 171496; Morrison et al., European Patent Application 173494 (published Mar. 5, 1986); Neuberger et al., PCT Application WO 86/01533, (published Mar. 13, 1986); Kudo et al., European Patent Application 184187; Morrison et al., European Patent Application 173494; Sahagan et al., J. Immunol. 137:1066-1074 (1986); Robinson et al., International Patent Publication PCT/US86/02269; Liu et al., Proc. Natl. Acad. Sci. USA 84:3439-3443 (1987); Sun et al., Proc. Natl. Acad. Sci. USA 84:214-218 (1987); Better et al., Science 240:1041-1043 (1988); and Harlow and Lane Antibodies: a Laboratory Manual Cold Spring Harbor Laboratory (1988)). These references are incorporated herein by reference for such disclosures.

In some embodiments the DP₂ ligand is a small molecule compound. In some embodiments the DP₂ ligand is a small molecule modulator of the DP₂ receptor. In one aspect, the DP₂ ligand is a small molecule antagonist of the DP₂ receptor. In one aspect, the DP₂ ligand is a small molecule agonist of the DP₂ receptor. In some embodiments, the DP₂ ligand is a natural endogenous ligand of the DP₂ receptor. Natural endogenous ligands of the DP₂ receptor include, but are not limited to, PGD₂, Δ¹²PGJ₂, 9α11βPGF₂, 13,14-dihydro-15-keto-PGD₂, or 15-deoxy-Δ^{12,14}PGD₂. In other embodiments, the ligand is an exogenous ligand.

Examples of small molecule DP₂ antagonists include, and are not limited to, compounds disclosed in U.S. provisional application No. 61/031,310; International patent application no. PCT/US09/35174; U.S. provisional application No. 60/985,919; International patent application no. PCT/US08/82056; U.S. provisional application No. 60/985,913; International patent application no. PCT/US08/82082; U.S. provisional application No. 61/025,597; International patent application no. PCT/US09/32495; International patent application no. PCT/US09/32499; U.S. application Ser. No. 12/362,439; U.S. provisional application No. 61/110,496; U.S. provisional application No. 61/028,804; International patent application no. PCT/US09/33961; U.S. provisional application No. 61/041,869; International patent application no. PCT/US09/38291; U.S. provisional application No. 61/078,311; International patent application no. PCT/US09/49621; International patent application no. PCT/US09/49631; U.S. application Ser. No. 12/497,343; U.S. provisional application No. 61/101,074; International patent application no. PCT/US09/58655; International patent application no. PCT/US09/58663; Ser. No. 12/568,571; U.S. provisional application No. 61/054,093; U.S. provisional application No. 61/107,638; International patent application no. PCT/US09/44219; U.S. provisional application No. 61/075,242; International patent application no. PCT/US09/48327; U.S. provisional application No. 61/101,964; International patent application no. PCT/US09/59256; U.S. provisional application No. 61/103,872; International patent application no. PCT/US09/59891; U.S. provisional application No. 61/115,259; International patent application no. PCT/US09/64630; U.S. provisional application No. 61/112,044; International patent application no. PCT/US09/63439; International patent application no. PCT/US09/63438; U.S. application Ser. No. 12/613,424; each of which is herein incorporated by reference in their entirety.

In one aspect, the DP₂ ligand is: ramatroban (Baynas, BAY u3405), AZD1981, ODC9101 (OC459), OC499, OC1768, OC2125, OC2184, QAV680, MLN6095, AP768, [2′-(3-benzyl-1-ethyl-ureidomethyl)-6-methoxy-4′-trifluoromethyl-biphenyl-3-yl]-acetic acid, {3-[2-tert-Butylsulfanylmethyl-4-(2,2-dimethyl-propionylamino)-phenoxy]-4-methoxy-phenyl}-acetic acid, TM30642, TM30643, TM30089, TM27632, TM3170, candesartan, as well as compounds disclosed in Pettipher et al, Nature Reviews Drug Discovery, vol. 6, 313-325, 2007; Medina et al., Annual Reports in Medicinal Chemistry, Vol. 41, 221-235, 2006, each of which is herein incorporated by reference).

In some embodiments, the DP₂ ligand is a peptide that can bind to the DP₂ receptor site. In other embodiments, the DP₂ ligand is a peptidomimetic analog of a peptide ligand. Peptide ligands include, but are not limited to, members of random peptide libraries; (see, e.g., Lam et al., 1991, Nature 354:82-84; Houghten et al., 1991, Nature 354:84-86), and combinatorial chemistry-derived molecular libraries made of D- and/or L-configuration amino acids, phosphopeptides (including, but not limited to, members of random or partially degenerate, directed phosphopeptide libraries; see, e.g., Songyang et al., 1993, Cell 72:767-778), recombinant (e.g., phage display libraries), and in vitro translation-based libraries. Examples of chemically synthesized peptide libraries are described in Fodor et al., 1991, Science 251:767-773; Houghten et al., 1991, Nature 354:84-86; Lam et al., 1991, Nature 354:82-84; Medynski, 1994, Bio/Technology 12:709-710; Gallop et al., 1994, J. Medicinal Chemistry 37(9):1233-1251; Ohlmeyer et al., 1993, Proc. Natl. Acad. Sci. USA 90:10922-10926; Erb et al., 1994, Proc. Natl. Acad. Sci. USA 91:11422-11426; Houghten et al., 1992, Biotechniques 13:412; Jayawickreme et al., 1994, Proc. Natl. Acad. Sci. USA 91:1614-1618; Salmon et al., 1993, Proc. Natl. Acad. Sci. USA 90:11708-11712; PCT Publication No. WO 93/20242; and Brenner and Lerner, 1992, Proc. Natl. Acad. Sci. USA 89:5381-5383. Examples of phage display libraries are described in Scott & Smith, 1990, Science 249:386-390; Devlin et al., 1990, Science, 249:404-406; Christian, et al., 1992, J. Mol. Biol. 227:711-718; Lenstra, 1992, J. Immunol. Meth. 152:149-157; Kay et al., 1993, Gene 128:59-65; and PCT Publication No. WO 94/18318 dated Aug. 18, 1994.

Peptidomimetic ligands include compounds from a benzodiazepine library (see e.g., Bunin et al., 1994, Proc. Natl. Acad. Sci. USA 91:4708-4712) that can be adapted for use for the methods disclosed herein, peptoid libraries disclosed in, e.g., Simon et al., 1992, Proc. Natl. Acad. Sci. USA 89:9367-9371 and peptidomimetics in which the amide functionalities in peptides have been permethylated to generate a chemically transformed combinatorial library (Ostresh et al. 1994, Proc. Natl. Acad. Sci. USA 91:11138-11142).

In further embodiments, the DP₂ ligand is an oligonucleotide or an aptamer. In some other embodiments, the DP₂ ligand is a nucleic acid.

### Labels

The methods and compositions disclosed herein make use of DP₂ ligands that include a label or tag. A label or tag allows for the detection of the labelled or tagged DP₂ ligand. A label or tag allows for the detection of DP₂ receptors in a biological sample when bound to a labelled or tagged DP₂ ligand. By label is meant a molecule that can be directly (i.e., a primary label) or indirectly (i.e., a secondary label) detected; for example a label can be visualized and/or measured or otherwise identified so that its presence or absence can be known. A labelled or tagged DP₂ ligand also allows for the detection of DP₂ receptors in a biological sample. A compound can be directly or indirectly conjugated to a label which provides a detectable signal, e.g. radioisotopes, fluorescers, enzymes, antibodies, particles such as magnetic particles, chemiluminescers, or specific binding molecules, etc. Specific binding molecules include pairs, such as biotin and streptavidin, digoxin and antidigoxin etc. Examples of labels include, but are not limited to, optical fluorescent and chromogenic dyes including labels, label enzymes and radioisotopes.

Labels include: a) isotopic labels, which may be radioactive or heavy isotopes; b) magnetic labels, electrical labels, thermal labels; c) colored labels, optical labels including luminescent, phosphorous and fluorescent dyes or moieties; and d) binding partners. Labels also include enzymes (e.g. horseradish peroxidase, etc.) and magnetic particles.

Labels include optical labels such as fluorescent dyes or moieties. Fluorophores are either “small molecule” fluors, or proteinaceous fluors (e.g. green fluorescent proteins and all variants thereof).

Suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™, Texas Red, IAEDANS, EDANS, BODIPY FL, LC Red 640, Cy 5, Cy 5.5, LC Red 705 and Oregon green. Suitable optical dyes are described in the 1996 Molecular Probes Handbook by Richard P. Haugland, hereby expressly incorporated by reference for such disclosure. Suitable fluorescent labels also include, but are not limited to, green fluorescent protein (GFP; Chalfie, et al., Science 263(5148):802-805, 1994); and EGFP; Clontech—Genbank Accession Number U55762), blue fluorescent protein (BFP; Quantum Biotechnologies, Inc.; Stauber, R. H. Biotechniques 24(3):462-471 (1998); Heim, R. and Tsien, R. Y. Curr. Biol. 6:178-182 (1996)), enhanced yellow fluorescent protein (EYFP; Clontech Laboratories, Inc.), luciferase (Ichiki, et al., J. Immunol. 150(12):5408-5417 (1993)), β-galactosidase (Nolan, et al., Proc Natl Acad Sci USA 85(8):2603-2607 (April 1988)) and Renilla (WO 92/15673; WO 95/07463; WO 98/14605; WO 98/26277; WO 99/49019; U.S. Pat. No. 5,292,658; U.S. Pat. No. 5,418,155; U.S. Pat. No. 5,683,888; U.S. Pat. No. 5,741,668; U.S. Pat. No. 5,777,079; U.S. Pat. No. 5,804,387; U.S. Pat. No. 5,874,304; U.S. Pat. No. 5,876,995; and U.S. Pat. No. 5,925,558).

In some embodiments, labels include: Alexa-Fluor dyes (Alexa Fluor 350, Alexa Fluor 430, Alexa Fluor 488, Alexa Fluor 546, Alexa Fluor 568, Alexa Fluor 594, Alexa Fluor 633, Alexa Fluor 660, Alexa Fluor 680), Cascade Blue, Cascade Yellow and R-phycoerythrin (PE) (Molecular Probes) (Eugene, Oreg.), FITC, Rhodamine, and Texas Red (Pierce, Rockford, Ill.), Cy5, Cy5.5, Cy7 (Amersham Life Science, Pittsburgh, Pa.). Tandem conjugate protocols for Cy5PE, Cy5.5PE, Cy7PE, Cy5.5APC, Cy7APC are known. In one aspect, quantitation of fluorescent probe conjugation is assessed to determine degree of labeling. In some embodiments the fluorescent label is conjugated to an aminodextran linker that is conjugated to a DP₂ ligand. Additional labels are available from commercial sources such as BD Biosciences, Beckman Coulter, AnaSpec, Invitrogen, Cell Signaling Technology, Millipore, eBioscience, Caltag, Santa Cruz Biotech, Abcam and Sigma-Aldrich.

In some embodiments, the fluorescent label is a GFP, a Renilla, Ptilosarcus, or Aequorea species of GFP.

Fluorescent labels that are attached to DP₂ ligands include dyes chosen for immunofluoroscence that are excited by light of one wavelength, usually blue or green, and emit light of a different wavelength in the visible spectrum. The most common fluorescent dyes are fluorescein, which emits green light, Texas Red and Peridinn chlorophyll protein (PerCP), which emit red light, and rhodamine and phycoerythrin (PE) which emit orange/red light. By using selective filters, only the light coming from the dye or fluorochrome used is detected in the fluorescence microscope. This technique can be used to detect DP₂ receptors in a biological sample.

The recent development of the confocal fluorescent microscope, which uses computer-aided techniques to produce an ultrathin optical section of a cell or tissue, gives very high resolution immunofluorescence microscopy without the need for elaborate sample preparation. The resolution of the confocal microscope can be further increased using low-intensity illumination so that two photons are required to excite the fluorochrome. A pulsed laser beam is used, and only when it is focused into the focal plane of the microscope is the intensity sufficient to excite fluorescence. In this way the fluorescence emission itself can be restricted to the optical section. In one embodiment, the use of a confocal fluorescent microscope allows for analysis of biological samples without any sample preparation.

In one embodiment, time-lapse video microscopy, in which sensitive digital video cameras record the movement of fluorescently labeled molecules in cell membranes, is used to assay a biological sample. Cell-surface DP₂ receptors can be fluorescently labeled in two main ways. One is by the binding of fluorochrome-labeled Fab fragments of antibodies specific for the DP₂ receptor; the other is by generating a fusion protein, in which the ligand of interest has been attached to one of a family of florescent proteins obtained from jellyfish like GFP. In some embodiments, the fluorescent labels include red, blue, cyan or yellow fluorescent proteins.

Fluorophore and chromophore labeled ligands are prepared from standard moieties. Since antibodies and other proteins absorb light having wavelengths up to about 310 nm, the fluorescent moieties is selected to have substantial absorption at wavelengths above 310 nm and preferably above 400 nm. A variety of suitable fluorescers and chromophores are described by Stryer, Science, 162:526 (1968) and Brand, L. et al., Annual Review of Biochemistry, 41:843-868 (1972), which are hereby incorporated by reference for such disclosure. The DP₂ ligands are labeled with fluorescent chromophore groups by conventional procedures such as those disclosed in U.S. Pat. Nos. 3,940,475, 4,289,747, and 4,376,110, which are hereby incorporated by reference for such disclosure.

One group of fluorescers having a number of the desirable properties described above are the xanthene dyes, which include the fluoresceins derived from 3,6-dihydroxy-9-henylxanthhydrol and resamines and rhodamines derived from 3,6-diamino-9-phenylxanthydrol and lissanime rhodamine B. The rhodamine and fluorescein derivatives of 9-o-carboxyphenylxanthhydrol have a 9-o-carboxyphenyl group. Fluorescein compounds having reactive coupling groups such as amino and isothiocyanate groups such as fluorescein isothiocyanate and fluorescamine are readily available. Another group of fluorescent compounds are the naphthylamines, having an amino group in the α- or β-position. In one aspect, DP₂ ligands are labeled with flurochromes or chromophores by the procedures described by Goding, J. W. (MONOCLONAL ANTIBODIES: PRINCIPLES AND PRACTICE. New York: Academic Press (1983) pp 208-249)

In some embodiments, chemiluminescers such as luciferin are attached to the DP₂ ligand (See, e.g., U.S. Pat. No. 5,098,828, for synthesis and methods of detection, incorporated by reference herein).

In some embodiments, the DP₂ ligand comprises a secondary detectable label. A secondary label is one that is indirectly detected; for example, a secondary label can bind or react with a primary label for detection, can act on an additional product to generate a primary label (e.g. enzymes), etc. Secondary labels include, but are not limited to, one of a binding partner pair; chemically modifiable moieties; nuclease inhibitors, enzymes such as horseradish peroxidase, alkaline phosphatases, luciferases, etc.

In some embodiments, the secondary label is a binding partner pair. In one aspect, the label is a hapten or antigen, which will bind its binding partner. For example, suitable binding partner pairs include, but are not limited to: antigens (such as proteins (including peptides) and small molecules) and antibodies (including fragments thereof (FAbs, etc.)); proteins and small molecules, including biotin/streptavidin; enzymes and substrates or inhibitors; other protein-protein interacting pairs; receptor-ligands; and carbohydrates and their binding partners. Nucleic acid-nucleic acid binding proteins pairs are contemplated. Binding partner pairs include, but are not limited to, biotin (or imino-biotin) and streptavidin, digeoxinin and Abs, and Prolinx™ reagents.

In some embodiments, the binding partner pair comprises an antigen and an antibody that will specifically bind to the antigen. By “specifically bind” herein is meant that the partners bind with specificity sufficient to differentiate between the pair and other components or contaminants of the system. The binding should be sufficient to remain bound under the conditions of the assay, including wash steps to remove non-specific binding. In some embodiments, the dissociation constants of the pair will be less than about 10⁻⁴ to 10⁻⁹ M⁻¹, with less than about 10⁻⁵ to 10⁻⁹ M⁻¹ being preferred and less than about 10⁻⁷ to 10⁻⁹ M⁻¹ being particularly preferred.

In some embodiment, the secondary label is a chemically modifiable moiety. In this embodiment, labels comprising reactive functional groups are incorporated into the molecule to be labeled. The functional group is then subsequently labeled (e.g. either before or after the assay) with a primary label. Suitable functional groups include, but are not limited to, amino groups, carboxy groups, maleimide groups, oxo groups and thiol groups. For example, primary labels containing amino groups are attached to secondary labels comprising amino groups, for example using known linkers; for example, homo- or hetero-bifunctional linkers (see 1994 Pierce Chemical Company catalog, technical section on cross-linkers, pages 155-200, incorporated herein by reference for such disclosure).

In some embodiments, multiple fluorescent labels are employed in the methods and compositions disclosed herein. In some embodiments, each label is distinct and distinguishable from other labels.

Antibody-label conjugation is performed using standard procedures or by using protein-protein/protein-dye cross-linking kits from Molecular Probes (Eugene, Oreg.).

In some embodiments, antibodies are labeled with quantum dots as disclosed by Chattopadhyay, P. K. et al. Quantum dot semiconductor nanocrystals for immunophenotyping by polychromatic flow cytometry. Nat. Med. 12, 972-977 (2006). Quantum dot labels are commercially available through Invitrogen, http://probes.invitrogen.com/products/qdot/.

Quantum dot labeled antibodies are used alone or they are employed in conjunction with organic fluorochrome-conjugated antibodies to increase the total number of labels available. As the number of labeled antibodies increase so does the ability for subtyping known cell populations. In some embodiments, antibodies are labeled using chelated or caged lanthanides as disclosed by Erkki, J. et al. Lanthanide chelates as new fluorochrome labels for cytochemistry. J. Histochemistry Cytochemistry, 36:1449-1451, 1988, and U.S. Pat. No. 7,018,850, entitled Salicylamide-Lanthanide Complexes for Use as Luminescent Markers. Other methods of detecting fluorescence include: Quantum dot methods (see, e.g., Goldman et al., J. Am. Chem. Soc. (2002) 124:6378-82; Pathak et al. J. Am. Chem. Soc. (2001) 123:4103-4; and Remade et al., Proc. Natl. Sci. USA (2000) 18:553-8) as well as confocal microscopy.

In some embodiments, the DP₂ ligands are labeled with tags suitable for Inductively Coupled Plasma Mass Spectrometer (ICP-MS) as disclosed in Tanner et al. Spectrochimica Acta Part B: Atomic Spectroscopy, 2007 March; 62(3):188-195; Ornatsky et al, Translational Oncogenomics (2006):1, 1-9; Ornatsky et al, Multiple Cellular Antigen Detection by ICP-MS, J. Imm. Methods 308 (2006) 68-76; and Lou et al., Polymer-Based Elemental Tags for Sensitive Bioassays, Angew. Chem. Int. Ed., (2007) 46, 6111-6114.

In one aspect, the DP₂ ligand comprises an enzyme label. By enzyme label is meant an enzyme that may be reacted in the presence of a label enzyme substrate that produces a detectable product. Enzyme labels include, and are not limited to, phosphatases or peroxidases covalently linked to a DP₂ ligand. Suitable enzyme labels include, but are not limited to, horseradish peroxidase, alkaline phosphatase and glucose oxidase. The presence of the enzyme label is generally revealed through the enzyme's catalysis of a reaction with a label enzyme substrate, producing an identifiable product that is detected and measured. The identifiable product may be a color change, detected with the naked eye or by a spectrophotometric technique, or the signal may be conversion of the substrate to a product that is detected by fluorescence. Such products may be opaque, such as the reaction of horseradish peroxidase with tetramethyl benzedine, and may have a variety of colors. Other label enzyme substrates, such as Luminol (available from Pierce Chemical Co.), have been developed that produce fluorescent reaction products. Methods for identifying label enzymes with label enzyme substrates are well known in the art and many commercial kits are available. Examples and methods for the use of various label enzymes are described in Savage et al., Previews 247:6-9 (1998), Young, J. Virol. Methods 24:227-236 (1989).

In one aspect, the DP₂ ligand comprises a radioisotope/radiolabel. By radioisotope is meant any radioactive molecule. Suitable radioisotopes include, but are not limited to ¹⁴C, ³H, ³²P, ³³P, ³⁵S, ¹²⁵I, ¹³¹I, ¹³N, ¹⁵O, ¹⁸F, ⁵⁷Co, ^{99m}Tc and ⁵¹Cr. The radiolabel is attached to the DP₂ ligand by covalent linkage. The incorporation of radioisotopes/radiolabels into the DP₂ ligand is accomplished using known techniques. For example, see Wensel and Meares, Radioimmunoimaging and Radioimmunotherapy, Elsevier, New York (1983), which is hereby incorporated by reference for such disclosure. See also, D. Colcher et al., “Use of Monoclonal Antibodies as Radiopharmaceuticals for the Localization of Human Carcinoma Xenografts in Athymic Mice”, Meth. Enzymol. 121: 802-816 (1986), which is hereby incorporated by reference for such disclosure. Tritium labeling procedures are described in U.S. Pat. No. 4,302,438, which is hereby incorporated by reference for such disclosure. Iodinating, tritium labeling, and ³⁵S labeling procedures for monoclonal antibodies are described by Goding, J. W. (MONOCLONAL ANTIBODIES: PRINCIPLES AND PRACTICE. New York: Academic Press (1983) pp 208-249), which are hereby incorporated by reference for such disclosure. Other procedures for iodinating ligands, such as antibodies, or binding portions thereof, probes, are described by Hunter and Greenwood, Nature 144:945 (1962), David et al., Biochemistry 13:1014-1021 (1974), and U.S. Pat. Nos. 3,867,517 and 4,376,110, which are hereby incorporated by reference for such disclosure. Procedures for ^{99m}Tc-labeling are described by Rhodes, B. et al. in Burchiel, S. et al. (eds.), Tumor Imaging: The Radioimmunochemical Detection of Cancer, New York: Masson 111-123 (1982) and the references cited therein, which are hereby incorporated by reference for such disclosure. Procedures suitable for ¹¹¹In-labeling of ligands are described by Hnatowich, D. J. et al., J. Immul. Methods, 65:147-157 (1983), Hnatowich, D. et al., J. Applied Radiation, 35:554-557 (1984), and Buckley, R. G. et al., F.E.B.S. 166:202-204 (1984), which are hereby incorporated by reference for such disclosure.

In one aspect, DP2 ligands include a radiolabel. In such a case scintillation counting is used. In such a case, PMN that have been exposed to the radiolabelled DP2 ligand are isolated and radioactivity of the bound ligands is measured.

In some embodiments, positron emitting isotopes detectable by a positron emission tomography (“PET”) scanner are attached to a ligand. Examples of positron emitting isotopes include radioisotopes with short half lives such as ¹¹C (˜20 min), ¹³N (˜10 min), ¹⁵O (˜2 min), and ¹⁸F (˜110 min).

Methods for labeling of proteins with radioisotopes are known in the art. For example, such methods are found in Ohta et al., (1999) Molec. Cell 3:535-541.

In some embodiments, DP₂ ligands are labeled with an NMR-active isotope label such as the ¹⁹F atom, or the ¹⁵N atom, or a plurality of such atoms.

In one aspect, the DP₂ ligands are labeled with an electron paramagnetic resonance (EPR) sensitive label. Examples of electron paramagnetic resonance (EPR) sensitive labels include, but are not limited to, a nitroso group.

In some embodiments, the labels are indirectly detected, that is, the tag is a partner of a binding pair. By “partner of a binding pair” is meant one of a first and a second moiety, wherein the first and the second moiety have a specific binding affinity for each other. Suitable binding pairs include, but are not limited to, antigens/antibodies (for example, digoxigenin/anti-digoxigenin, dinitrophenyl (DNP)/anti-DNP, dansyl-X-anti-dansyl, Fluorescein/anti-fluorescein, lucifer yellow/anti-lucifer yellow, and rhodamine anti-rhodamine), biotin/avidin (or biotin/streptavidin) and calmodulin binding protein (CBP)/calmodulin. Other suitable binding pairs include polypeptides such as the FLAG-peptide [Hopp et al., BioTechnology, 6:1204-1210 (1988)]; the KT3 epitope peptide [Martin et al., Science, 255: 192-194 (1992)]; tubulin epitope peptide [Skinner et al., J. Biol. Chem., 266:15163-15166 (1991)]; and the T7 gene 10 protein peptide tag [Lutz-Freyermuth et al., Proc. Natl. Acad. Sci. USA, 87:6393-6397 (1990)] and the antibodies each thereto.

In some embodiments, a partner of one binding pair is also a partner of another binding pair. For example, an antigen (first moiety) binds to a first antibody (second moiety) that, in turn, is an antigen for a second antibody (third moiety). It will be further appreciated that such a circumstance allows indirect binding of a first moiety and a third moiety via an intermediary second moiety that is a binding pair partner to each.

In some embodiments, a partner of a binding pair comprises a label, as described herein. It will further be appreciated that this allows for a tag to be indirectly labeled upon the binding of a binding partner comprising a label. Attaching a label to a tag that is a partner of a binding pair, as just described, is referred to herein as “indirect labeling”.

The production of tag-polypeptides by recombinant means when the tag is also a polypeptide is described below. Production of tag-labeled proteins is well known in the art and kits for such production are commercially available (for example, from Kodak and Sigma). Examples of tag labeled proteins include, but are not limited to, a Flag-polypeptide and His-polypeptide. Methods for the production and use of tag-labeled proteins are found, for example, in Winston et al., Genes and Devel. 13:270-283 (1999).

Biotinylation of target molecules and substrates is well known, for example, a large number of biotinylation agents are known, including amine-reactive and thiol-reactive agents, for the biotinylation of proteins, nucleic acids, carbohydrates, carboxylic acids; see chapter 4, Molecular Probes Catalog, Haugland, 6th Ed. 1996, hereby incorporated by reference for such disclosure. A biotinylated substrate can be attached to a biotinylated component via avidin or streptavidin. Similarly, a large number of haptenylation reagents are also known.

In some embodiments, the tag is functionalized with chemically reactive groups such as thiols, amines, carboxyls, etc. to facilitate covalent attachment. The covalent attachment of the tag may be either direct or via a linker In one embodiment, the linker is a relatively short coupling moiety, which is used to attach the molecules. A coupling moiety may be synthesized directly onto a component of the tag and contains at least one functional group to facilitate attachment of the tag. Alternatively, the coupling moiety may have at least two functional groups, which are used to attach a functionalized component to a functionalized tag, for example. In an additional embodiment, the linker is a polymer. In this embodiment, covalent attachment is accomplished either directly, or through the use of coupling moieties from the component or tag to the polymer. In some embodiments, the covalent attachment is direct, that is, no linker is used. In this embodiment, the component preferably contains a functional group such as a carboxylic acid that is used for direct attachment to the functionalized tag. It should be understood that the component and tag may be attached in a variety of ways, including those listed above. In some embodiments, the tag is attached to the amino or carboxy terminus of the polypeptide.

In some embodiments, the tag is functionalized to facilitate covalent attachment, as is generally outlined above. Thus, a wide variety of tags are commercially available which contain functional groups, including, but not limited to, isothiocyanate groups, amino groups, haloacetyl groups, maleimides, succinimidyl esters, and sulfonyl halides, all of which may be used to covalently attach the tag to a second molecule, as is described herein. The choice of the functional group of the tag will depend on the site of attachment to either a linker, as outlined above or a component of the invention. Thus, for example, for direct linkage to a carboxylic acid group of a protein, amino modified or hydrazine modified tags will be used for coupling via carbodiimide chemistry, for example using 1-ethyl-3-(3-dimethylaminopropyl)-carbodiimide (EDAC) as is known in the art (see Set 9 and Set 11 of the Molecular Probes Catalog, supra; see also the Pierce 1994 Catalog and Handbook, pages T-155 to T-200, both of which are hereby incorporated by reference).

### Detection

In practicing the methods disclosed herein, the detection of DP₂ receptors in a biological sample is carried out using at least one analytical instrument/device. The type of analytical instrument(s) depends on the technique that is being used to detect the DP₂ receptors in the biological sample. In one aspect, the detection of DP₂ receptors is carried out using automated systems. In any case, the detection of DP₂ receptors in a biological sample is performed according to standard techniques and protocols established in the art or adapted from the art.

DP₂ receptors are detected and/or quantified by any method or combination of methods that detect and/or quantitates the presence of protein receptors. Such methods include, but are not limited to, radioimmunoassay (RIA) or enzyme linked immunoabsorbance assay (ELISA), immunohistochemistry, immunofluorescent histochemistry with or without confocal microscopy, reversed phase assays, homogeneous enzyme immunoassays, and related non-enzymatic techniques, Western blots, whole cell staining, immunoelectronmicroscopy, nucleic acid amplification, gene array, protein array, mass spectrometry, patch clamp, 2-dimensional gel electrophoresis, differential display gel electrophoresis, microsphere-based multiplex protein assays, label-free cellular assays and flow cytometry, etc. U.S. Pat. No. 4,568,649 describes ligand detection systems, which employ scintillation counting. Cell readouts for proteins and other cell determinants can be obtained using fluorescent or otherwise tagged reporter molecules.

In some embodiments, the methods of detecting further include steps of isolating a particular type of cell or tissue. Exemplary methods to isolate a particular cell from other cells in a population include, but are not limited to, Fluorescent Activated Cell Sorting (FACS) as described, for example, in Shapiro, Practical Flow Cytometry, 3rd edition Wiley-Liss; (1995), density gradient centrifugation, or manual separation using micromanipulation methods with microscope assistance. Exemplary cell separation devices include, without limitation, a Beckman JE-6 centrifugal elutriation system, Beckman Coulter EPICS ALTRA computer-controlled Flow Cytometer-cell sorter, Modular Flow Cytometer from Cytomation, Inc., Coulter counter and channelyzer system, density gradient apparatus, cytocentrifuge, Beckman J-6 centrifuge, EPICS V dual laser cell sorter, or EPICS PROFILE flow cytometer. In some embodiments, a tissue or population of cells is removed by surgical techniques.

When using fluorescent labeled components in the methods and compositions disclosed herein, different types of fluorescent monitoring systems, e.g., Cytometric measurement device systems, are used in some instances. In some embodiments, flow cytometric systems are used or systems dedicated to high throughput screening, e.g. 96 well or greater microtiter plates. Methods of performing assays on fluorescent materials are known in the art and are described in, e.g., Lakowicz, J. R., Principles of Fluorescence Spectroscopy, New York: Plenum Press (1983); Herman, B., Resonance energy transfer microscopy, in: Fluorescence Microscopy of Living Cells in Culture, Part B, Methods in Cell Biology, vol. 30, ed. Taylor, D. L. & Wang, Y.-L., San Diego: Academic Press (1989), pp. 219-243; Turro, N. J., Modern Molecular Photochemistry, Menlo Park: Benjamin/Cummings Publishing Col, Inc. (1978), pp. 296-361.

In one aspect, fluorescence in a sample is measured using a fluorimeter. In general, excitation radiation, from an excitation source having a first wavelength, passes through excitation optics. The excitation optics cause the excitation radiation to excite the sample. In response, fluorescent proteins in the sample emit radiation that has a wavelength that is different from the excitation wavelength. Collection optics then collect the emission from the sample. The device can include a temperature controller to maintain the sample at a specific temperature while it is being scanned. According to one embodiment, a multi-axis translation stage moves a microtiter plate holding a plurality of samples in order to position different wells to be exposed. The multi-axis translation stage, temperature controller, auto-focusing feature, and electronics associated with imaging and data collection can be managed by an appropriately programmed digital computer. The computer also can transform the data collected during the assay into another format for presentation. In one aspect, known robotic systems and components are used.

Other methods of detecting fluorescence include, e.g., Quantum dot methods (see, e.g., Goldman et al., J. Am. Chem. Soc. (2002) 124:6378-82; Pathak et al. J. Am. Chem. Soc. (2001) 123:4103-4; and Remade et al., Proc. Natl. Sci. USA (2000) 18:553-8) as well as confocal microscopy. In general, flow cytometry involves the passage of individual cells through the path of a laser beam. The light scattering of the beam and excitation of any fluorescent molecules attached to, or found within, the cell is detected by photomultiplier tubes to create a readable output, e.g. size, granularity, or fluorescent intensity.

In one aspect, the detecting, sorting, and/or isolating step of the methods disclosed herein comprises fluorescence-activated cell sorting (FACS) techniques, where FACS is used to select cells (e.g. neutrophils) from the population containing a particular surface marker (e.g. DP₂ receptor), or the selection step can entail the use of magnetically responsive particles as retrievable supports for target cell capture and/or background removal. A variety of FACS systems are known in the art (see e.g., WO99/54494, filed Apr. 16, 1999; U.S. Ser. No. 20010006787, filed Jul. 5, 2001, each incorporated herein by reference).

In some embodiments, a FACS cell sorter (e.g. a FACSVantage™ Cell Sorter, Becton Dickinson Immunocytometry Systems, San Jose, Calif.) is used to sort and collect cells based on their expression of the DP₂ receptor and interaction with a DP₂ ligand comprising a fluorescent label.

In some embodiments, a biological sample is first contacted with fluorescent-labeled DP₂ ligand. In such an embodiment, the amount of fluorescent-labeled DP₂ ligand bound on each cell in the biological sample is measured by passing droplets containing the cells through the cell sorter. By imparting an electromagnetic charge to droplets containing the positive cells (i.e. cells that express the DP₂ receptor and bind a fluorescent-labeled DP₂ ligand), the cells are separated from other cells. The positively selected cells are harvested in sterile collection vessels. These cell-sorting procedures are described in detail, for example, in the FACSVantage™ Training Manual, with particular reference to sections 3-11 to 3-28 and 10⁻¹ to 10-17, which is hereby incorporated by reference in its entirety.

In some embodiment, cell analysis by flow cytometry on the basis of the presence of the DP₂ receptor is combined with a determination of other flow cytometry readable outputs, such as the presence of surface markers, granularity and cell size to provide a correlation between the presence of the DP₂ receptor and other cell qualities measurable by flow cytometry for single cells.

In another embodiment, cells expressing the DP₂ receptor (i.e. positive cells) are sorted using magnetic separation of cells based on the presence of the DP₂ receptor. In such separation techniques, cells to be positively selected are first contacted with labelled DP₂ ligand. The cells are then contacted with retrievable particles (e.g., magnetically responsive particles) that are coupled with the labelled DP₂ ligand. The cell-labelled DP₂ ligand-particle complex is then physically separated from non-positive or non-labeled cells, for example, using a magnetic field. When using magnetically responsive particles, the positive or labeled cells are retained in a container using a magnetic field while the negative cells are removed. These and similar separation procedures are described, for example, in the Baxter Immunotherapy Isolex training manual which is hereby incorporated in its entirety.

In some embodiments, the detection of DP₂ receptors on cells is performed with an Inductively Coupled Plasma Mass Spectrometer (ICP-MS). A DP₂ ligand that has been labeled with a specific element binds to the DP₂ receptor. In one aspect, the DP₂ ligand is labelled with an element selected from Au, Sm, Eu, and Tb. When the cell is introduced into the ICP, it is atomized and ionized. The elemental composition of the cell, including the labeled DP₂ ligand that is bound to the DP₂ receptors, is measured. The presence and intensity of the signals corresponding to the labels on the DP₂ ligand indicates the level of the DP₂ receptor on that cell (Tanner et al. Spectrochimica Acta Part B: Atomic Spectroscopy, (2007), 62(3):188-195; Journal of Immunological Methods, Volume 308, Issues 1-2, 20 Jan. 2006, Pages 68-76).

In some embodiments confocal microscopy is used to detect DP₂ receptors expressed on cells. Confocal microscopy relies on the serial collection of light from spatially filtered individual specimen points, which is then electronically processed to render a magnified image of the specimen. The signal processing involved in confocal microscopy has the additional capability of detecting labeled ligands within single cells, accordingly in this embodiment the cells can be labeled with one or more labelled ligands (at least one of which is a labelled DP₂ ligand). In some embodiments the labelled ligands used in connection with confocal microscopy are antibodies conjugated to fluorescent labels, however other labelled ligands, such as other peptides or nucleic acids are also possible.

In some embodiments, a “In-Cell Western Assay” is used. In such an assay, cells are initially grown in standard tissue culture flasks using standard tissue culture techniques. Once grown to optimum confluency, the growth media is removed and cells are washed and trypsinized. The cells are then counted and volumes sufficient to transfer the appropriate number of cells are aliquoted into microwell plates (e.g., Nunc™ 96 Microwell™ plates). The individual wells are then grown to optimum confluency in complete media whereupon the media is replaced with serum-free media. At this point controls are untouched, but experimental wells are incubated with a modulator, e.g. an endotoxin and/or neutrophil chemoattractant/activating ligands. After incubation with the modulator cells are fixed and stained with labeled ligands (e.g. antibodies to the DP₂ receptor, antibodies for the identification of neutrophils). Once the cells are labeled, the plates are scanned using an imager such as, but not limited to, the Odyssey Imager (LiCor, Lincoln Nebr.) using techniques described in the Odyssey Operator's Manual v1.2., which is hereby incorporated. Data obtained by scanning of the multi-well plate are analyzed and activation profiles determined as described below.

In some embodiments, the detecting is by high pressure liquid chromatography (HPLC), for example, reverse phase HPLC, and in a further aspect, the detecting is by mass spectrometry.

Flexible hardware and software allow instrument adaptability for multiple applications. The software program modules allow creation, modification, and running of methods. The system diagnostic modules allow instrument alignment, correct connections, and motor operations. Customized tools, labware, and liquid, particle, cell and organism transfer patterns allow different applications to be performed. Databases allow method and parameter storage. Robotic and computer interfaces allow communication between instruments.

In some embodiment, the methods of detecting include the use of liquid handling components. The liquid handling systems include robotic systems comprising any number of components. In some embodiments, any or all of the steps outlined herein are automated. In one aspect, the systems are completely or partially automated.

There are a wide variety of components that can be used, including, but not limited to, one or more robotic arms; plate handlers for the positioning of microplates; automated lid or cap handlers to remove and replace lids for wells on non-cross contamination plates; tip assemblies for sample distribution with disposable tips; washable tip assemblies for sample distribution; 96 well loading blocks; cooled reagent racks; microtiter plate pipette positions (optionally cooled); stacking towers for plates and tips; and computer systems.

Fully robotic or microfluidic systems include automated liquid-, particle-, cell- and organism-handling including high throughput pipetting to perform all steps of screening applications. This includes liquid, particle, cell, and organism manipulations such as aspiration, dispensing, mixing, diluting, washing, accurate volumetric transfers; retrieving, and discarding of pipet tips; and repetitive pipetting of identical volumes for multiple deliveries from a single sample aspiration. These manipulations are cross-contamination-free liquid, particle, cell, and organism transfers. This instrument performs automated replication of microplate samples to filters, membranes, and/or daughter plates, high-density transfers, full-plate serial dilutions, and high capacity operation.

In some embodiments, chemically derivatized particles, plates, cartridges, tubes, magnetic particles, or other solid phase matrix with specificity to the assay components are used. The binding surfaces of microplates, tubes or any solid phase matrices include non-polar surfaces, highly polar surfaces, modified dextran coating to promote covalent binding, antibody coating, affinity media to bind fusion proteins or peptides, surface-fixed proteins such as recombinant protein A or G, nucleotide resins or coatings, and other affinity matrix are used.

In some embodiments, platforms for multi-well plates, multi-tubes, holders, cartridges, minitubes, deep-well plates, microfuge tubes, cryovials, square well plates, filters, chips, optic fibers, beads, and other solid-phase matrices or platform with various volumes are accommodated on an upgradeable modular platform for additional capacity. This modular platform includes a variable speed orbital shaker, and multi-position work decks for source samples, sample and reagent dilution, assay plates, sample and reagent reservoirs, pipette tips, and an active wash station. In some embodiments, the methods of detecting include the use of a plate reader.

In some embodiments, thermocycler and thermoregulating systems are used for stabilizing the temperature of heat exchangers such as controlled blocks or platforms to provide accurate temperature control of incubating samples from 0° C. to 100° C.

In some embodiments, interchangeable pipet heads (single or multi-channel) with single or multiple magnetic probes, affinity probes, or pipetters robotically manipulate the liquid, particles, cells, and organisms. Multi-well or multi-tube magnetic separators or platforms manipulate liquid, particles, cells, and organisms in single or multiple sample formats.

In some embodiments, the instrumentation will include a detector, which can be a wide variety of different detectors, depending on the labels and assay. In some embodiments, useful detectors include a microscope(s) with multiple channels of fluorescence; plate readers to provide fluorescent, ultraviolet and visible spectrophotometric detection with single and dual wavelength endpoint and kinetics capability, luminescence, quenching, two-photon excitation, and intensity redistribution; CCD cameras to capture and transform data and images into quantifiable formats; and a computer workstation.

In some embodiments, the robotic apparatus includes a central processing unit which communicates with a memory and a set of input/output devices (e.g., keyboard, mouse, monitor, printer, etc.) through a bus. Again, as outlined below, this may be in addition to or in place of the CPU for the multiplexing devices. The general interaction between a central processing unit, a memory, input/output devices, and a bus is known in the art. Thus, a variety of different procedures, depending on the experiments to be run, are stored in the CPU memory.

These robotic fluid handling systems utilize any number of different reagents, including buffers, reagents, samples, washes, assay components such as label probes, etc.

### Diagnostic Kits/Articles of Manufacture

For use in the diagnostic applications described herein, kits and articles of manufacture are also provide herein. Such kits can comprise a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. The containers are formed from any acceptable material including, e.g., glass or plastic.

For example, the container(s) can comprise one or more compounds described herein, optionally in a composition or in combination with another agent as disclosed herein. The container(s) optionally have a sterile access port (for example the container can be an intravenous solution bag or a vial having a stopper pierceable by a hypodermic injection needle). Such kits optionally comprising a compound with an identifying description or label or instructions relating to its use in the methods described herein.

A kit will typically comprise one or more additional containers, each with one or more of various materials (such as reagents, optionally in concentrated form, and/or devices) desirable from a commercial and user standpoint for use of a compound described herein. Non-limiting examples of such materials include, but are not limited to, buffers, diluents, filters, needles, syringes; carrier, package, container, vial and/or tube labels listing contents and/or instructions for use, and package inserts with instructions for use. A set of instructions will also typically be included.

A label can be on or associated with the container. A label can be on a container when letters, numbers or other characters forming the label are attached, molded or etched into the container itself; a label can be associated with a container when it is present within a receptacle or carrier that also holds the container, e.g., as a package insert. A label can be used to indicate that the contents are to be used for a specific therapeutic application. The label can also indicate directions for use of the contents, such as in the methods described herein.

An in vitro diagnostic kit for detection of DP₂ receptors in a biological sample comprises a DP₂ ligand, where the DP₂ ligand comprises a detectable label. The labelled DP₂ ligand is provided in the kit in a lyophilized or crystal form. In some embodiments, the kit further comprises a liquid that is used to reconstitute the DP₂ ligand. In some embodiments, the DP₂ ligand is provided packaged in an aqueous medium. The kit comprises instructions for the detection of DP₂ ligands in a biological sample from a human.

In one aspect, an in vitro diagnostic kit for detection of DP₂ receptors in a biological sample comprises a DP2 selective antibody and a fluorescent second antibody for use in FACs analysis. The kit comprises instructions for the detection of DP2 in a biological sample from a human.

### EXAMPLES

These examples are provided for illustrative purposes only and not to limit the scope of the claims provided herein.

### Example 1

### Synthesis of Fluorescent Tagged DP2 Receptor Antagonists

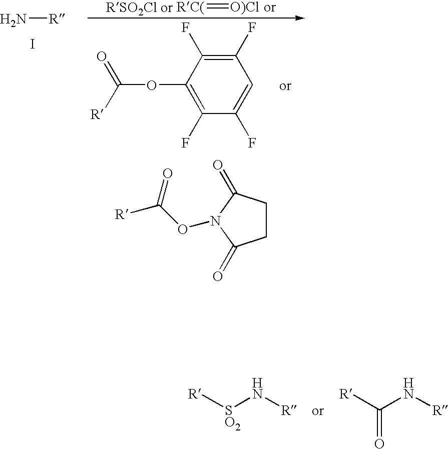

Fluorescent labeled DP2 antagonists are prepared by standard coupling reactions including coupling the amine precursor I (the DP2 antagonist) with commercially available active esters or sulfonyl chlorides derivatives of the fluorescent tag. Standard coupling reactions, techniques and materials, including those found in March, ADVANCED ORGANIC CHEMISTRY 4^{th} Ed., (Wiley 1992); Carey and Sundberg, ADVANCED ORGANIC CHEMISTRY 4^{th} Ed., Vols. A and B (Plenum 2000, 2001), and Green and Wuts, PROTECTIVE GROUPS IN ORGANIC SYNTHESIS 3^{rd} Ed., (Wiley 1999).

Other reagents used to couple amine precursor I with fluorescent tags include, but are not limited to, coupling reagent such as EDC EDC, DCC, BOP, HATU or the like. If needed, a suitable base is used to facilitate the coupling reaction. In certain cases the amine precursor 1 includes an ester function that is then subsequently hydrolyzed to the carboxylic acid in a separate step using aqueous LiOH or NaOH and an organic solvent such as THF or methanol.

TABLE 1
Fluorescent-Labeled antagonists of DP2 as diagnostic agents.

| | |
|---|---|
| | 1 |
| 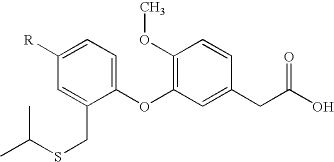 | |
| | 2 |
| 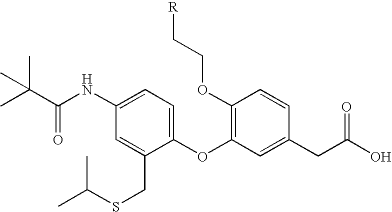 | |
| | 3 |
| 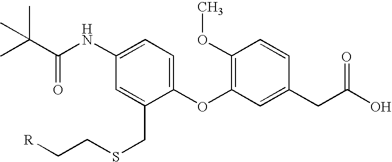 | |

| Compound no. | R | structure |
|---|---|---|
| 1-1 | Dansyl-NH | 1 |
| 1-2 | Texas red-NH | 1 |
| 1-3 | Alexa Fluor 350-NH | 1 |
| 1-4 | Alexa Fluor 488-NH | 1 |
| 1-5 | Alexa Fluor 546-NH | 1 |
| 1-6 | Alexa Fluor 555-NH | 1 |
| 1-7 | Alexa Fluor 647-NH | 1 |
| 1-8 | Cy3-NH | 1 |
| 1-9 | Cy5-NH | 1 |
| 1-10 | Cy7-NH | 1 |
| 2-1 | Dansyl-NH | 2 |
| 2-2 | Texas red-NH | 2 |
| 2-3 | Alexa Fluor 350-NH | 2 |
| 2-4 | Alexa Fluor 488-NH | 2 |
| 2-5 | Alexa Fluor 546-NH | 2 |
| 2-6 | Alexa Fluor 555-NH | 2 |
| 2-7 | Alexa Fluor 647-NH | 2 |
| 2-8 | Cy3-NH | 2 |
| 2-9 | Cy5-NH | 2 |
| 2-10 | Cy7-NH | 2 |
| 3-1 | Dansyl-NH | 3 |
| 3-2 | Texas red-NH | 3 |
| 3-3 | Alexa Fluor 350-NH | 3 |
| 3-4 | Alexa Fluor 488-NH | 3 |
| 3-5 | Alexa Fluor 546-NH | 3 |
| 3-6 | Alexa Fluor 555-NH | 3 |
| 3-7 | Alexa Fluor 647-NH | 3 |
| 3-8 | Cy3-NH | 3 |
| 3-9 | Cy5-NH | 3 |
| 3-10 | Cy7-NH | 3 |

### Example 2

### Method for Inducing the Expression of DP₂ in a Human Blood Neutrophil Population and the Detection of the DP₂ Expression by Selective Antibody and Fluorescent Second Antibody FACs Analysis

Blood was drawn from consenting human volunteers into heparin containing vacutainer tubes and used within one hour of draw. 500 μl aliquots of blood were transferred to 1.5 ml eppendorf tubes. The blood was stimulated by the addition of either vehicle (phosphate buffered saline-PBS), 1 μg/ml LPS, 100 nM fMLP or 1 μg/ml LPS plus 100 nM fMLP. When blood was treated with LPS plus fMLP, the samples were primed by the addition of the LPS for 30 minutes at 37° C. before the addition of the fMLP. The blood samples were then incubated for either 4 hours or 22 hours at 37° C. The red blood cells were lysed by the addition of ammonium chloride lysing solution (155 mM NH₄Cl, 10 mM KHCO₃, 0.1 mM Na₂EDTA, pH 7.2) and incubation at room temperature for 15 minutes. The white blood cells were pelleted at 1200 rpm for 5 minutes and washed once with PBS The cells were then stained with an anti-CD16 antibody conjugated to the fluorochrome PC5 (Beckman Coulter, clone 3G8) and an anti-DP₂ antibody conjugated to the fluorochrome R-phycoerytherin (PE) (Miltenyi Biotech, clone BM16) for 30 minutes on ice. The cells were washed twice with cold PBS and resuspended in cold cytofix (BD Biosciences). DP₂ expression on the neutrophils was analyzed using a FACSCalibur (BD Biosciences) and gating on the CD16 high, side scatter (SSC) high cells.

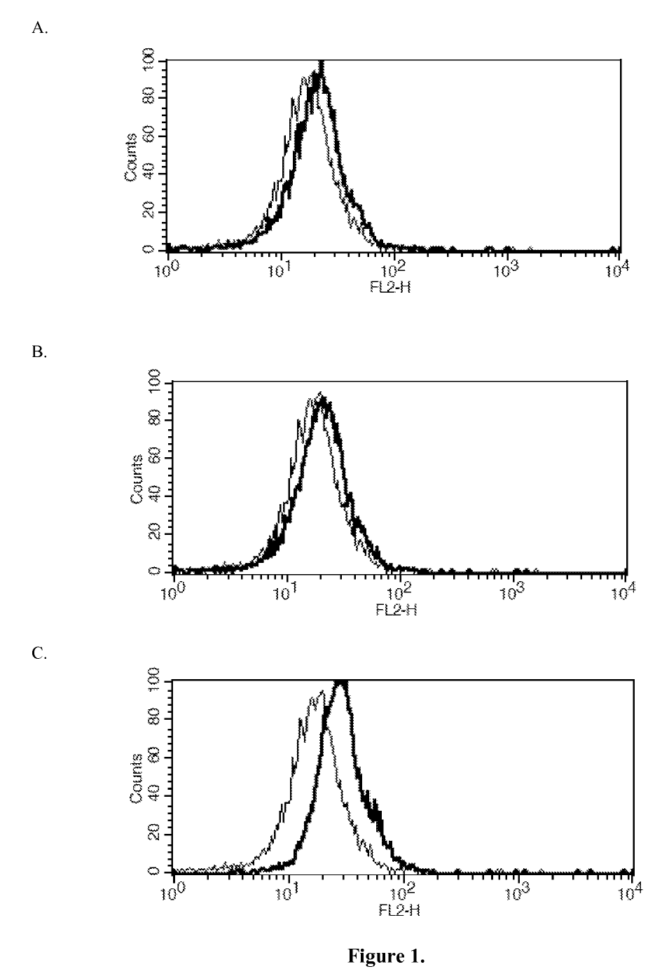

FIG. 1 presents the induction of expression of DP₂ on human peripheral blood neutrophils following 4 hour incubation with LPS and/or fMLP. DP₂ expression on neutrophils from human whole blood treated for 4 hours with either (A) 1 μg/ml LPS (B) 100 nM fMLP or (C) 1 μg/ml LPS plus 100 nM fMLP. In each histogram, the thin line represents DP₂ staining on neutrophils from vehicle treated blood and the bold line represents DP₂ staining on neutrophils from stimulated blood.

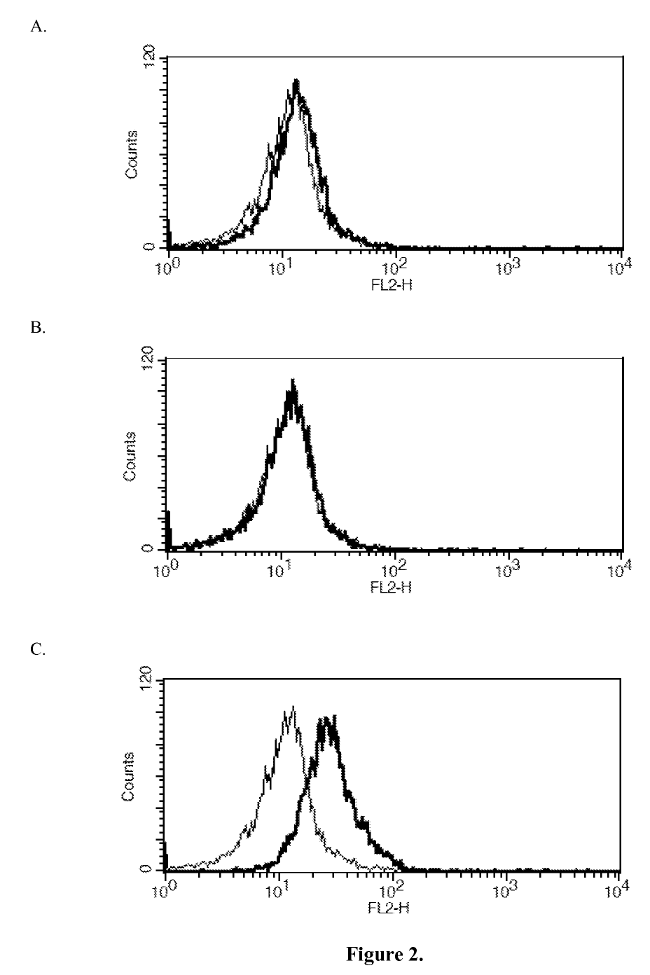

FIG. 2 presents the induction of expression of DP₂ on human peripheral blood neutrophils following 22 hour incubation with LPS and/or fMLP. DP₂ expression on neutrophils from human whole blood treated for 22 hours with either (A) 1 μg/ml LPS (B) 100 nM fMLP or (C) 1 μg/ml LPS plus 100 nM fMLP. In each histogram, the thin line represents DP₂ staining on neutrophils from vehicle treated blood and the bold line represents DP₂ staining on neutrophils from stimulated blood

The examples and embodiments described herein are for illustrative purposes only and various modifications or changes suggested to persons skilled in the art are to be included within the spirit and purview of this application and scope of the appended claims. The section headings used herein are for organizational purposes only and are not to be construed as limiting the subject matter described.

What is claimed is:

1. A method of identifying a human for treatment with a DP₂ antagonist comprising:
detecting DP₂ receptors in a transformed biological sample from a human using at least one analytical instrument.

2. The method of claim 1, wherein:
the method detects DP₂ receptors expressed on polymorphonuclear leukocytes in the transformed biological sample from the human.

3. The method of claim 2, wherein:
the human has at least one symptom of an inflammatory disease or condition.

4. The method of claim 3, further comprising:
classifying the inflammatory disease or condition as a PGD₂-dependent or a PGD₂-mediated disease or condition based on the detection of DP₂ receptors in the transformed biological sample from the human; or
classifying the human as eligible to receive treatment with a DP₂ antagonist based on the detection of DP₂ receptors in the transformed biological sample from the human.

5. The method of claim 1, wherein:
the transformed biological sample is produced by transforming the DP₂ receptors in a biological sample into DP₂ receptors that are detectable using at least one analytical instrument; and
detecting DP₂ receptors in the transformed biological sample from the human using at least one analytical instrument employs a technique selected from the group consisting of: Fluorescence-activated Cell Sorting (FACS), confocal, western blot, flow cytometry, fluorescence microscopy, scintillation counting, quantititative polymerase chain reaction, Magnetic-activated Cell Sorting (MACS).

6. The method of claim 3, wherein:
the inflammatory disease or condition is a respiratory disease or condition; and
the method detects DP₂ receptors expressed on neutrophils in the transformed biological sample from the human.

7. The method of claim 6, wherein:
the respiratory disease or condition is selected from asthma, chronic obstructive pulmonary disease (COPD), pulmonary hypertension, interstitial lung fibrosis, adult respiratory distress syndrome, and airway constriction; and
the biological sample from the human is a biological fluid or biological tissue sample from or around the lungs.

8. The method of claim 7, further comprising:
classifying the human as eligible to receive therapy for the respiratory disease or condition based on the detection of DP₂ receptors expressed on neutrophils in the biological sample from the human.

9. The method of claim 8, wherein:
detecting DP₂ receptors in the biological sample from the human using at least one analytical instrument comprises transforming the DP₂ receptors in the biological sample into DP₂ receptors that are detectable using the least one analytical instrument; and
detecting DP₂ receptors in the biological sample from the human using at least one analytical instrument employs a technique selected from the group consisting of: Fluorescence-activated Cell Sorting (FACS), confocal, western blot, flow cytometry, fluorescence microscopy, scintillation counting, quantititative polymerase chain reaction, magnetic-activated Cell Sorting (MACS).

10. The method of claim 9, wherein:
the classifying further comprises comparing the number of neutrophils that express DP₂ receptors in the biological sample to the number of neutrophils that express DP₂ receptors in a whole blood sample from the human.

11. A method of identifying a human for treatment with a DP₂ antagonist comprising:
transforming DP₂ receptors in a biological sample from a human into DP₂ receptors that are detectable using at least one analytical instrument; and
a) identifying cells in the biological sample from a human that express the DP₂ receptor using at least one analytical instrument;
b) quantifying the number of cells in the biological sample from the human that express the DP₂ receptor using at least one analytical instrument; or
c) identifying and quantifying the number of cells in the biological sample from the human that express the DP₂ receptor using at least one analytical instrument.

12. The method of claim 11, wherein:
the cells that are identified, quantified, or identified and quantified that express the DP₂ receptor using at least one analytical instrument are neutrophils; and
the human has at least one symptom of an inflammatory disease or condition.

13. The method of claim 12, further comprising:
classifying the human as eligible to receive treatment with a DP₂ antagonist based on the detection of DP₂ receptors expressed on neutrophils in the biological sample from the human.

14. The method of claim 13, wherein:
detecting DP₂ receptors in the biological sample from the human using at least one analytical instrument employs a technique selected from the group consisting of: Fluorescence-activated Cell Sorting (FACS), confocal, western blot, flow cytometry, fluorescence microscopy, scintillation counting, quantititative polymerase chain reaction, magnetic-activated Cell Sorting (MACS).

15. The method of claim 13, wherein:
the classifying further comprises comparing the number of neutrophils that express DP₂ receptors in the biological sample from the human from the area of disease activity to the number of neutrophils that express DP₂ receptors in a whole blood sample from the human.

16. A method of monitoring the clinical efficacy of a DP₂ antagonist in a human comprising comparing: (1) the detection of DP₂ receptors in a first biological sample from a human using at least one analytical instrument prior to the administration of a DP₂ antagonist to the human, with (2) the detection of DP₂ receptors in a second biological sample from the human using at least one analytical instrument after the administration of the DP₂ antagonist to the human; wherein the first biological sample and the second biological sample are the same, and the first biological sample and the second biological sample comprise polymorphonuclear leukocytes.

17. The method of claim 16, wherein:
the method compares the detection of DP₂ receptors expressed on polymorphonuclear leukocytes in the biological samples; and
a reduction of the number of polymorphonuclear leukocytes expressing the DP₂ receptor that are detected in the second biological sample relative to the number of polymorphonuclear leukocytes expressing the DP₂ receptor that are detected in the first biological samples indicates a positive response to the DP₂ antagonist.

18. The method of claim 16, wherein:
the method compares the detection of DP₂ receptors expressed on neutrophils in the biological samples; and
a reduction of the number of neutrophils expressing the DP₂ receptor that are detected in the second biological sample relative to the number of neutrophils expressing the DP₂ receptor that are detected in the first biological samples indicates a positive response to the DP₂ antagonist.

19. The method of claim 16, wherein the human has at least one symptom of an inflammatory disease or condition.

20. The method of claim 16, wherein the detection of DP₂ receptor in the first biological sample and the second biological sample employs a technique selected from the group consisting of: Fluorescence-activated Cell Sorting (FACS), confocal, western blot, flow cytometry, fluorescence microscopy, quantitative polymerase chain reaction, scintillation counting, magnetic-activated Cell Sorting (MACS).
